(19) United States
(12) Patent Application Publication
(10) Pub. No.: US 2025/0127579 A1
(43) Pub. Date: Apr. 24, 2025

(54) Robot Command Input Based on Image-Plane Intersections for Transcatheter Robotic Therapies and Other Uses

(71) Applicant: Project Moray, Inc., Belmont, CA (US)

(72) Inventors: Vritika Agarwal, New York, NY (US); Susheel Kodali, Hastings On Hudson, NY (US); Nathan Wong, Oakland, CA (US); Erik Stauber, Albany, CA (US)

(73) Assignee: Project Moray, Inc,, Belmont, CA (US)

(21) Appl. No.: 18/885,974

(22) Filed: Sep. 16, 2024

Related U.S. Application Data
(60) Provisional application No. 63/538,602, filed on Sep. 15, 2023.

(51) Int. Cl. A61B 34/37; A61B 8/08; A61B 8/12; A61B 17/00; A61B 17/128; A61B 34/00; A61B 34/10; A61B 34/20; A61B 34/30; A61B 90/00; A61M 25/01

(52) U.S. Cl. CPC A61B 34/37; A61B 8/0883; A61B 8/12; A61B 17/1285; A61B 34/74; A61B 2017/00243; A61B 2034/107; A61B 2034/2063; A61B 2034/301; A61B 2090/365; A61B 2090/367; A61B 2090/378; A61M 2025/0166

(57) ABSTRACT

Medical therapeutic and diagnostic devices, systems, and methods help guide transcatheter heart therapies with reference to 3D image data by viewing a plurality of planar images that are generated digitally from the 3D image data. Input used by the clinical team to manipulate the image planes within the worksite may be used to direct movement of the therapeutic or diagnostic tool itself by setting a target axis of a tool in alignment with a line of intersection between two imaging planes, by using a point at which three image planes intersect as a target point, and by transmitting commands to a transcatheter robot system supporting the tool to move in the worksite into alignment with the target axis and target point.

### CROSS-REFERENCE TO RELATED APPLICATION DATA

The present application claims the benefit under 35 USC § 119(e) of U.S. Provisional Appln. No. 63/538,602 filed Sep. 15, 2023; the full disclosure which is incorporated herein by reference in its entirety for all purposes.

The subject matter of the present application is related to that of PCT/US2023/016751 filed Mar. 29, 2023; which claims the benefit of U.S. Provisional Appln. Nos. 63/325,068 filed Mar. 29, 2022 and 63/403,096 filed Sep. 1, 2022; the full disclosures which are incorporated herein by reference in their entirety for all purposes.

### FIELD OF THE INVENTION

In general, the present invention provides improved articulating devices, articulating systems, and methods for using elongate articulate bodies and other tools such as medical robots, transcatheter therapy and diagnostic systems, cardiovascular catheters, and the like; as well as for using improved image processing devices, systems and methods. Embodiments of the inventions described herein may find particularly beneficial use for guiding medical robots with reference to three-dimensional (“3D”) ultrasound, computed tomography (“CT”) x-ray, magnetic resonance imaging (“MRI”), fluoroscopic, and other image data, including for display and image-guided movement of a portion of a transcatheter medical robot that has been inserted into a patient.

### BACKGROUND OF THE INVENTION

Diagnosing and treating disease often involve accessing internal tissues of the human body, and open surgery is often the most straightforward approach for gaining access to those internal tissues. Although open surgical techniques have been highly successful, they can impose significant trauma to collateral tissues.

To help avoid the trauma associated with open surgery, a number of minimally invasive surgical access and treatment technologies have been developed. Interventional therapies are among the most successful minimally invasive approaches. An interventional therapy often makes use of elongate flexible catheter structures that can be advanced along a vascular pathway through the network of blood vessel lumens extending throughout the body. Alternative technologies have been developed to advance diagnostic and/or therapeutic devices through the trachea and into the bronchial passages of the lung. While generally limiting trauma to the patient, catheter-based endoluminal therapies can be challenging, in-part due to the difficulty in accessing and accurately aligning with a target tissue using an instrument traversing a tortuous luminal path. Alternative minimally invasive surgical technologies include surgical robotics, and robotic systems for manipulation of flexible catheter bodies from outside the patient have also been proposed. Some of those prior robotic catheter systems have met with challenges, possibly because of the difficulties in effectively integrating large and complex robotic systems into clinical catheter labs, respiratory treatment suites, and the like. While the potential improvements to surgical accuracy make these efforts alluring, the capital equipment costs and overall burden to the healthcare system of these large, specialized systems is also a concern.

A range of technologies for controlling the shape and directing the movement of catheters have been proposed, including catheter assemblies with opposed pullwires for use in robotic articulating structures. Such structures often seek to provide independent lateral bending along perpendicular bending axes using two pairs of orthogonally oriented pullwires. As more fully explained in co-assigned PCT Publn. No. WO 2020/123671, which was filed on Dec. 11, 2019, and entitled “HYBRID-DIMENSIONAL, AUGMENTED REALITY, AND/OR REGISTRATION OF USER INTERFACE; AND SIMULATION SYSTEMS FOR ROBOTIC CATHETERS AND OTHER USES,” the full disclosure of which is incorporated herein by reference, new fluid-driven and other robotic catheter systems can optionally be driven with reference to a 3D augmented reality display. While those advantageous drive systems, articulation control, and therapy systems will find a wide variety of applications for use by interventional and other doctors in guiding the movement of articulated therapy delivery systems within a patient, it would be beneficial to even further expand the capabilities of these compact and intuitive robotic systems.

Precise control of both manual and robotic interventional articulating structures can be complicated by the challenges of efficiently and accurately identifying the current position, orientation, and articulation state of flexible structures within an internal surgical site, as well as the desired position and orientation of the therapeutic tools carried by the articulating structure. Interventionalists often rely on multiple remote imaging modalities to plan and guide different aspects of the therapy, for example, viewing single-image-plane fluoroscopy images while accessing the heart and then viewing multi-plane and 3D echocardiography images to see and interact with the target tissue structures. Maintaining situational awareness and precise control of a complex interventional therapy in this environment can be a significant challenge.

In general, it would be beneficial to provide improved medical robotic and other articulating devices, systems, and methods. It would be particularly beneficial if these improved technologies could expand the capabilities and ease-of-use of image guidance systems for use in diagnostic and therapeutic interventional procedures, ideally by providing control and user-interaction technologies which help the clinical team to see and guide movement of structures within the patient.

### BRIEF SUMMARY OF THE INVENTION

The present invention generally provides improved medical therapeutic and diagnostic devices, systems, and methods. Exemplary methods help guide transcatheter heart therapies with reference to 3D image data, most often while viewing a plurality of echocardiographic (“echo”) or other image planes or “slices” through a worksite. The planar images may be generated digitally from the 3D image data, and the user may manipulate the image planes digitally using a graphical user interface (rather than relying solely on movement of an ultrasound probe or the like). The imaging system will often derive the planar images using multi-plane reconstruction (“MPR”) technology. The planar images may optionally be combined in a 3D workspace shown in a display, the workspace ideally also including a virtual 3D model of a diagnostic or therapeutic tool the user is positioning in the heart. Alignment or registration of the imaging data, virtual tool, and movement commands input by the user may be provided using multi-thread computer vision or image processing with a series of parent/child reference frames associated with the image planes, an image capture device, the tool, etc. In some embodiments, the input used by the clinical team to manipulate the image planes within the worksite may also be used to direct movement of the therapeutic or diagnostic tool itself, for example, by setting a target axis or trajectory of the tool in alignment with a line of intersection between two imaging planes, by using a point at which three image planes intersect as a target point, and by transmitting commands to a transcatheter robot system supporting the tool to move in the worksite into alignment with the target axis and target point.

In a first aspect, the invention provides a robot system for aligning a diagnostic or therapeutic robot with a target tissue in a three-dimensional (3D) workspace inside a patient body. The robot system is for use with an ultrasound system including a transducer for generating three-dimensional (“3D”) image data of the 3D workspace, a display for showing a plurality of planar images of the 3D workspace, an input, and a multi-plane reconstruction (“MPR”) module coupling the transducer and the input to the display so that the planar images on the display show the 3D data along imaging planes, with each planar image having an associated one of the image planes within the workspace. The MPR module is configured to facilitate manipulation of the imaging planes using the input. Advantageously, an intersection of the imaging planes can define an image-based position and an image-based orientation within the 3D workspace. The robot system comprises an articulating arm and a proximal driver, the articulating arm having a proximal end and a distal end with an axis therebetween. The proximal end can be coupled with the driver, and the distal end is configured for insertion into the 3D workspace within the patient. The driver is coupled with the processor of the ultrasound system so as to induce driving of the distal end of the articulating arm toward the image-based position and image-based orientation in response to the user manipulating the input of the imaging system.

A number of additional independent features may optionally be included to enhance the functionality of the devices, systems, and methods provided herein. For example, the robot system will often include a therapeutic or diagnostic tool supported by the distal end of the articulated arm, with suitable tools comprising transcatheter therapy tools such as a replacement valve, a valve repair tool such as a transcatheter edge-to-edge repair (“TEER”) clip, an annuloplasty tool such as an annuloplasty ring, a chordae repair tool such as an artificial chordae and associated anchors. Other suitable tools may comprise ablation tools for electrophysiology arrythmia therapies, occlusive devices for right or left atrial appendage closure (“LAAC”), atrial or septal defect closure, paravalvular leak closure, patent foramen ovale closure, and the like. Advantageously, an augmented reality (“AR”) module can couple the processor of the imaging system with the display, with the AR module configured to superimpose an image of a virtual version of the therapeutic or diagnostic tool on the display at the image-based position and orientation. The processor can induce driving of the actual tool, as sensed by the imaging system, as included in the 3D image data, and as shown in the display, into alignment with the virtual tool, as shown in the display (both of which may or may not be shown at the same time). Optionally, the driving of the actual tool may (but need not) be performed when the robotic arm is advanced axially into the patient.

Preferably, the robotic arm comprises a robotically articulated catheter, and the transducer of the imaging system comprises a transesophageal echocardiography (“TEE”) or intracardiac echocardiography (“ICE”) transducer. The robotic articulated catheter can be configured for use within the cardiovascular system, and may support (near its distal end) a tool configured for performing a transcatheter interventional therapy. For example, the tool may optionally comprise a transcatheter edge-to-edge repair (“TEER”) clip. Advantageously, a catheter trajectory planning module may be included and can be configured to display an AR catheter body extending from a guide sheath to the virtual tool. A trajectory modification input can also be included for altering an AR axis of the AR catheter body, with the trajectory modification input optionally allowing a clinician to move a location along the AR catheter body laterally along one or more of the imaging planes in the 3D workspace. Other trajectory modification input options may be provided, for example, to allow the clinician to change a position and/or orientation of a distal end of a virtual guide sheath, with the processor determining an alternative AR axis of the AR catheter body. An insertion control module can also be provided for varying a shape of the actual axis of the actual catheter body to induce following of the AR axis by the actual distal end during axial advancement of the catheter toward the target tissue. Optionally, the robotic articulated catheter may support and robotically move the ICE transducer within the patient.

The ultrasound system may generate an image data stream during use. The robot system may include a module that is coupled with the driver and that is configured to determine a pose of the transducer in the 3D workspace. The robot system may also include an image processing module coupling the processor of the ultrasound system with the driver. The image processing mode may be configured to identify poses of the image planes relative to the transducer in response to the image data stream. The driver can make use of the pose of the transducer and the poses of the image planes to provide movement of the distal end in response to the clinician's positioning of the image planes. Alternatively, the ultrasound system may transmit image plane pose data that identifies the poses of the image planes relative to the transducer (in addition to the image data stream). The processing of the pose data, generating of movement commands for the robotic arm, and any image processing associated with determining poses or commands may be performed on a robot processor circuitry that is coupled with the ultrasound system processor, or the processors may be integrated together or separated into a wide variety of alternative data architectures.

In another aspect, the invention provides a robot system for commanding movement of a robot system in a three-dimensional (3D) workspace shown in a display. The robot system comprises an elongate body having a proximal end and a distal end with an axis therebetween. A robotic driver is couplable with the proximal end of the elongate body. A processor is couplable to the driver, the processor configured for receiving (relative to a first image plane) a first command to move a second image plane represented in the display. The first image plane and second image plane can be disposed in the 3D workspace (often with a significant angle therebetween). The image planes may be represented in a display, for example, with the first image plane extending along an associated first window of the display and the second image plane extending along a second window of the display, the two windows in the display often being substantially coplanar. The processor can also be configured for receiving, relative to the second image plane, a second command to move the first image plane. The processor can be configured to determine a line of intersection between the first image plane and the second image plane in the 3D workspace, and to transmit a robot movement command to the driver so that the axis of the body moves toward alignment with the line of intersection in the 3D workspace.

In a method aspect, the invention provides a method for aligning a diagnostic or therapeutic robot with a target tissue in a three-dimensional (“3D”) workspace inside a patient body. The method comprises generating 3D image data of the 3D workspace and showing a plurality of planar images of the 3D workspace on a display. The transducer is coupled to the display so that the planar images on the display show the 3D data along associated imaging planes. The imaging planes are manipulated using the input, and an intersection of the imaging planes defines an image-based position and orientation within the 3D workspace. A distal end of an articulating arm in the 3D workspace is driven toward the image-based position and orientation in response to the user manipulating the input of the imaging system.

In another method aspect, the invention provides a method for commanding movement of a robot system in a three-dimensional (3D) workspace shown in a display. The robot system has a body with an axis, and the method comprises receiving, with a processor and relative to a first image plane, a first command to move a second image plane shown in the display. The first image plane and the second image plane are disposed in the 3D workspace and shown in the display, with the first image plane extending along an associated first window of the display and the second image plane extending along a second window of the display. The processor receives, relative to the second image plane, a second command to move the first image plane. The processor determines a line of intersection between the first image plane and the second image plane in the 3D workspace, and transmits a robot movement command to the robot so that the axis of the body moves toward alignment with the line of intersection in the 3D workspace.

The robot system optionally includes a therapeutic or diagnostic tool supported by a distal end of the body, and the method may optionally comprise superimposing an image of a virtual therapeutic or diagnostic tool on the display with the axis aligned at the line of intersection. The robot movement command may drive the tool, as shown in the display, into alignment with the virtual tool, as shown in the display (though both need not be seen at the same time), ideally when the robotic arm is advanced axially into the patient. The robot system may include 3D image data from the 3D workspace, and the second command to move the first image plane may cause reconstruction of a first 2D image along the first image plane from the 3D image data. The first 2D image may be shown in the first window of the display, and the first command to move the second image plane may lead to reconstruction of a second 2D image along the second image plane from the 3D image data, the second 2D image being shown in the second window of the display. Optionally, an input device may sense the first command being entered by a hand of a user. The first command may, for example, comprising a change (as shown in the second window), to a position of the line of intersection, or a change to an orientation of the line of intersection, or both. The input device may also sense the second command as entered by a hand of a user, the second command comprising a change, as shown in the first window, to a position of the line of intersection or a change to an orientation of the line of intersection.

### BRIEF DESCRIPTION OF THE DRAWINGS

FIG. 1 illustrates an interventional cardiologist performing a structural heart procedure with a robotic catheter system having a fluidic catheter driver slidably supported by a stand.

FIGS. 2 and 2A are perspective views of components of a robotic catheter system in which a catheter is removably mounted on a driver assembly, in which the driver assembly includes a driver encased in a sterile housing and supported by the stand, and in which the catheter is inserted distally into the patient through a guide sheath.

FIG. 3 schematically illustrates a robotic catheter system and transmission of signals between the components thereof so that input from a user induces a desired articulation.

FIGS. 4A and 4B schematically illustrate a data processing system architecture for a robotic catheter system and transmission of signals between a user interface, a motion controller, and embedded circuitry of a drive assembly.

FIG. 5 is a functional block diagram schematically illustrating software components and data flows of the motion controller of FIGS. 4A and 4B.

FIG. 6 is a functional block diagram schematically illustrating data processing components included in a single-use replaceable catheter and data flows between those components and the data processing components of a reusable driver assembly on which the catheter is mounted.

FIG. 7 is a perspective view of robotic catheter system and a clinical user of that system showing a 3D user input space and a 2D or 3D display space of the system with which the user interacts.

FIG. 8 schematically illustrates images included in the display space of FIG. 7, showing 2D and 3D image components represented in the display space and their associated reference frames as registered using the registration system of the robotic system data processor so that the user environment of the robotic system user interface presents a coherent 3D workspace to the user.

FIGS. 9A-9D illustrate optional image elements to be included in an exemplary hybrid 2D/3D display image of the user interface.

FIG. 10 is a block diagram schematically illustrating components and data transmission of a registration module, including a fluoro-based pose module, an echo-based pose module, and a sensor-based pose module.

FIG. 11 is a perspective view illustrating a C-arm or cone-beam fluoroscopy image acquisition system and a localization sensor or reference supported by a patient table.

FIG. 12 is a top view of a panel for positioning between a patient support and a patient, the panel having an exemplary array of Aruco fiducial markers.

FIG. 13 is a perspective view of an exemplary guide sleeve of a robotic toolset, the guide sleeve having an array of Aruco fiducial markers distributed axially and circumferentially with the individual markers bending to a substantially cylindrical arc-segment cross section along the outer profile of the guide sheath.

FIG. 14 is a screen shot from an image-based registration module including both an alignment module that aligns fluoroscopic image data with an internal therapy site using image data from a planar array of fiducial markers, and a fluoroscopy-based pose module that determines a ultrasound-based pose of a guide sheath using image data from a plurality of fiducial markers mounted to the guide sheath together with a CAD model of the guide sheath.

FIG. 15 illustrates an ultrasound multiplane reconstruction (MPR) display having a plurality of planes and a 3D image which can be registered with a workspace and displayed in the display system described herein.

FIG. 16 illustrates exemplary components of an ultrasound data acquisition system.

FIG. 17 is a functional block diagram of a multi-thread computer program for registering a plurality of interventional components having parent/child reference coordinate relationships using multi-mode image data; and FIGS. 17A-17E show portions and software modules of that program enlarged for ease of review.

FIG. 18 is a graphical user interface for the computer program of FIG. 32.

FIG. 19 illustrates an exemplary ultrasound system for use in embodiments of the image-based robotic input techniques described herein.

FIG. 20 is a functional block diagram schematically illustrating components of an exemplary robotic system that can be driven by image-based robotic input.

FIG. 21 is a screen shoot showing image based robotic input entered so as to manipulate image planes using the ultrasound system of FIG. 19.

FIGS. 22A-22D are screen shots of an auxiliary input of the robotic system of FIG. 20 showing driving of virtual (AR) and actual robotic components in response to manipulation of image planes of the ultrasound system of FIG. 19.

FIGS. 23A and 23B are perspective views of an auxiliary display output showing an AR catheter body coupling a guide to an AR tool pose based on image planes of an ultrasound system.

FIG. 24 schematically illustrates a method for sensing image plane poses of an ultrasound system using image processing.

FIGS. 25-31 illustrate aspects of an image processing or computer vision module that can be used to determine the poses of image plane within a worksite.

FIGS. 32A-35B graphically illustrate image plane geometry used herein to help show how poses of image planes can optionally be derived.

### DETAILED DESCRIPTION OF THE INVENTION

The improved devices, systems, and methods for robotic catheters and other systems described herein will find a wide variety of uses. The elongate articulated structures described herein will often be flexible, typically comprising catheters suitable for insertion in a patient body. The structures described herein will often find applications for diagnosing or treating the disease states of or adjacent to the cardiovascular system, the alimentary tract, the airways, the urogenital system, the neurovasculature, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with one or more (preferably multiple) degrees of freedom without having to resort to complex rigid linkages.

Exemplary systems and structures provided herein may be configured for insertion into the vascular system, the systems typically including a cardiac catheter and supporting a structural heart tool for repairing or replacing a valve of the heart, occluding an ostium or passage, or the like. Other cardiac catheter systems will be configured for diagnosis and/or treatment of congenital defects of the heart, or may comprise electrophysiology catheters configured for diagnosing or inhibiting arrhythmias (optionally by ablating a pattern of tissue bordering or near a heart chamber). Alternative applications may include use in steerable supports of image acquisition devices such as for trans-esophageal echocardiography (TEE), intra-coronary echocardiography (ICE), and other ultrasound techniques, endoscopy, and the like. Still further applications may make use of structures configured as interventional neurovascular therapies that articulate within the vasculature system which circulates blood through the brain, facilitating access for and optionally supporting stroke mitigation devices such as aneurism coils, thrombectomy structures (including those having structures similar to or derived from stents), neurostimulation leads, or the like.

Embodiments described herein may fully or partly rely on pullwires to articulate a catheter or other elongate flexible body. With or without pullwires, alternative embodiments provided herein may use balloon-like structures to effect at least a portion of the articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medical structures described herein will often have an articulated distal portion and an unarticulated proximal portion, which may significantly simplify initial advancement of the structure into a patient using standard catheterization techniques.

The medical robotic systems described herein will often include an input device, a driver, and a toolset configured for insertion into a patient body. The toolset will often (though will not always) include a guide sheath having a working lumen extending therethrough, and an articulated catheter (sometimes referred to herein as a steerable sleeve) or other robotic manipulator, and a diagnostic or therapeutic tool supported by the articulated catheter, the articulated catheter typically being advanced through the working lumen of the guide sheath so that the tool is at an internal therapy site. The user will typically input commands into the input device, which will generate and transmit corresponding input command signals. The driver will generally provide both power for and articulation movement control over the tool. Hence, somewhat analogous to a motor driver, the driver structures described herein will receive the input command signals from the input device and will output drive signals to the tool-supporting articulated structure so as to effect robotic movement of the tool (such as by inducing movement of one or more laterally deflectable segments of a catheter in multiple degrees of freedom). The drive signals may optionally comprise fluidic commands, such as pressurized pneumatic or hydraulic flows transmitted from the driver to the tool-supporting catheter along a plurality of fluid channels. Optionally, the drive signals may comprise mechanical, electromechanical, electromagnetic, optical, or other signals, with or without fluidic drive signals. Many of the systems described herein inducement movement using fluid pressure. Unlike many robotic systems, the robotic tool supporting structure will often (though not always) have a passively flexible portion between the articulated feature (typically disposed along a distal portion of a catheter or other tool manipulator) and the driver (typically coupled to a proximal end of the catheter or tool manipulator). The system may be driven while sufficient environmental forces are imposed against the tool or catheter to impose one or more bend along this passive proximal portion, the system often being configured for use with the bend(s) resiliently deflecting an axis of the catheter or other tool manipulator by 10 degrees or more, more than 20 degrees, or even more than 45 degrees.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transvers to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the curved outer surface of the cylinder between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

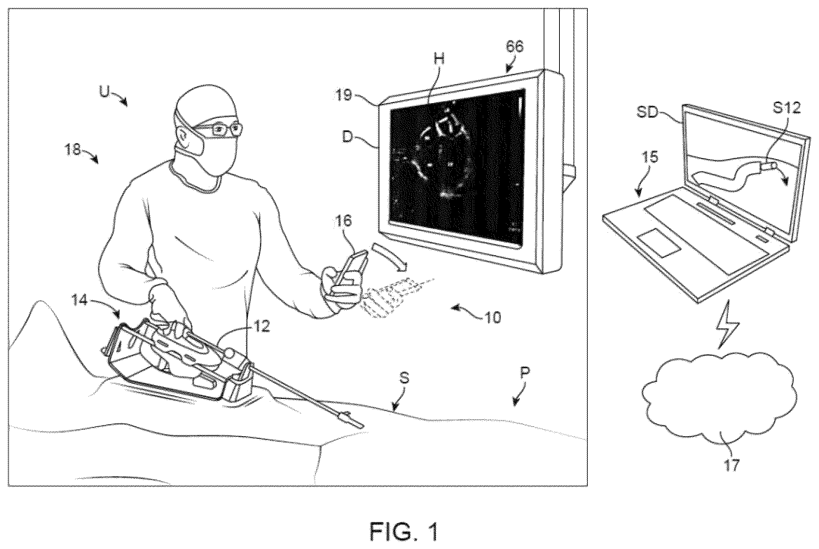

Referring first to FIG. 1, a system user U, such as an interventional cardiologist, uses a robotic catheter system 10 to perform a procedure in a heart H of a patient P. System 10 generally includes an articulated catheter 12, a driver assembly 14, and an input device 16. User U controls the position and orientation of a therapeutic or diagnostic tool mounted on a distal end of catheter 12 by entering movement commands into input 16, and optionally by axially moving the catheter relative to a stand of the driver assembly, while viewing an image of the distal end of the catheter and the surrounding tissue in a display D.

During use, catheter 12 extends distally from driver system 14 through a vascular access site S, optionally (though not necessarily) using an introducer sheath. A sterile field 18 encompasses access site S, catheter 12, and some or all of an outer surface of driver assembly 14. Driver assembly 14 will generally include components that power automated movement of the distal end of catheter 12 within patient P, with at least a portion of the power often being generated and modulated using hydraulic or pneumatic fluid flow. To facilitate movement of a catheter-mounted therapeutic tool per the commands of user U, system 10 will typically include data processing circuitry, often including a processor within the driver assembly. Regarding that processor and the other data processing components of system 10, a wide variety of data processing architectures may be employed. The processor, associated pressure and/or position sensors of the driver assembly, and data input device 16, optionally together with any additional general purpose or proprietary computing device (such as a desktop PC, notebook PC, tablet, server, remote computing or interface device, or the like) will generally include a combination of data processing hardware and software, with the hardware including an input, an output (such as a sound generator, indicator lights, printer, and/or an image display), and one or more processor board(s). These components are included in a processor system capable of performing the transformations, kinematic analysis, and matrix processing functionality associated with generating the valve commands, along with the appropriate connectors, conductors, wireless telemetry, and the like. The processing capabilities may be centralized in a single processor board, or may be distributed among various components so that smaller volumes of higher-level data can be transmitted. The processor(s) will often include one or more memory or other form of volatile or non-volatile storage media, and the functionality used to perform the methods described herein will often include software or firmware embodied therein. The software will typically comprise machine-readable programming code or instructions embodied in non-volatile media and may be arranged in a wide variety of alternative code architectures, varying from a single monolithic code running on a single processor to a large number of specialized subroutines, classes, or objects being run in parallel on a number of separate processor sub-units.

Referring still to FIG. 1, along with display D, a simulation display SD may present an image of an articulated portion of a simulated or virtual catheter S12 with a receptacle for supporting a simulated therapeutic or diagnostic tool. The simulated image shown on the simulation display SD may optionally include a tissue image based on pre-treatment imaging, intra-treatment imaging, and/or a simplified virtual tissue model, or the virtual catheter may be displayed without tissue. Simulation display SD may have or be included in an associated computer 15, and the computer will preferably be couplable with a network and/or a cloud 17 so as to facilitate updating of the system, uploading of treatment and/or simulation data for use in data analytics, and the like. Computer 15 may have a wireless, wired, or optical connection with input device 16, a processor of driver assembly 14, display D, and/or cloud 17, with suitable wireless connections comprising a Bluetooth™ connection, a WiFi connection, or the like. Preferably, an orientation and other characteristics of simulated catheter S12 may be controlled by the user U via input device 16 or another input device of computer 15, and/or by software of the computer so as to present the simulated catheter to the user with an orientation corresponding to the orientation of the actual catheter as sensed by a remote imaging system (typically a fluoroscopic imaging system, an ultra-sound imaging system, a magnetic resonance imaging system (MRI), or the like) incorporating display D and an image capture device 19. Optionally, computer 15 may superimpose an image of simulated catheter S12 on the tissue image shown by display D (instead of or in addition to displaying the simulated catheter on simulation display SD), preferably with the image of the simulated catheter being registered with the image of the tissue and/or with an image of the actual catheter structure in the therapy or surgical site. Still other alternatives may be provided, including presenting a simulation window showing simulated catheter SD on display D, including the simulation data processing capabilities of computer 15 in a processor of driver assembly 14 and/or input device 16 (with the input device optionally taking the form of a tablet) that can be supported by or near driver assembly 14, incorporating the input device, computer, and one or both of displays D, SD into a workstation near the patient, shielded from the imaging system, and/or remote from the patient, or the like.

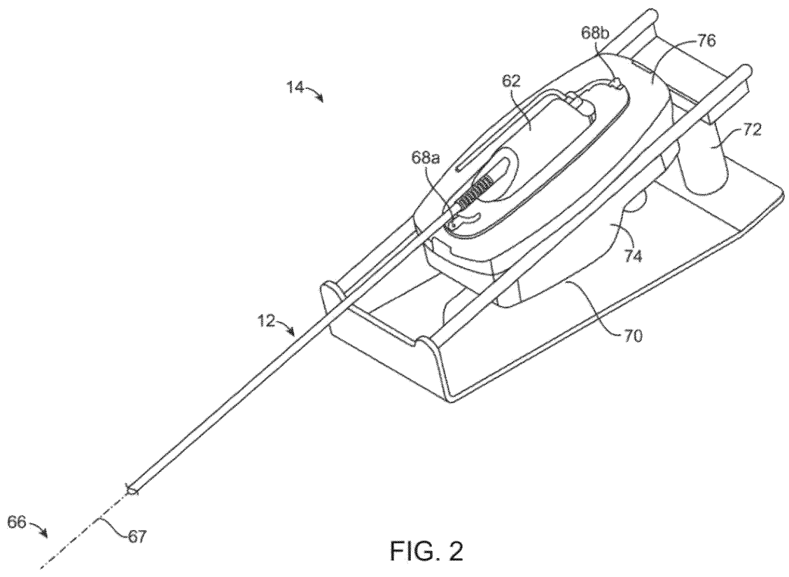

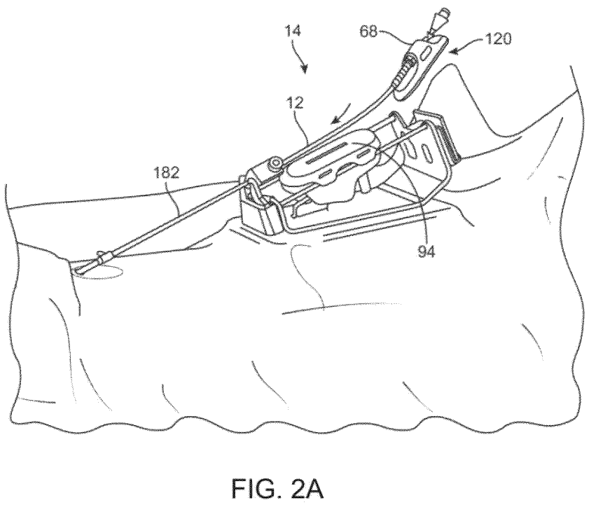

Referring now to FIG. 2, catheter 12 is removably mounted on exemplary driver assembly 14 for use. Catheter 12 has a proximal portion 22 and a distal portion 24 with an axis 26 extending therebetween. A proximal housing 28 of catheter 12 has an interface 30 that sealingly couples with an interface 32 of a driver 34 included in driver assembly 14 so that fluid drive channels of the driver are individually sealed to fluid channels of the catheter housing, allowing separate pressures to be applied to control the various degrees of freedom of the catheter. Driver 34 is contained within a sterile housing 36 of driver assembly 14. Driver assembly 14 also includes a support 38 or stand with rails extending along the axis 26 of the catheter, and the sterile housing, driver, and proximal housing of the catheter are movably supported by the rails so that the axial position of the catheter and the associated catheter drive components can move along the axis under either manual control or with powered robotic movement. Details of the sterile housing, the housing/driver interface, and the support are described in PCT Patent Publication No. WO 2019/195841, assigned to the assignee of the subject application, and filed on Apr. 8, 2019, the full disclosure of which is incorporated herein by reference.

Referring now to FIG. 2A, a guide sheath 182 is introduced into and advanced within the vasculature of the patient, optionally through an introducer sheath (though no introducer sheath may be used in alternate embodiments). Guide sheath 182 may optionally have a single pull-wire for articulation of a distal portion of the guide sheath, similar to the guide catheter used with the MitraClip™ mitral valve therapy system as commercially available from Abbott. Alternatively, the guide sheath may be an unarticulated tubular structure that can be held straight by the guidewire or a dilator extending within the lumen along the bend, or use of the guide sheath may be avoided. Regardless, when used the guide sheath will often be advanced manually by the user toward a surgical site over a guidewire, with the guide sheath often being advanced up the inferior vena cava (IVC) to the right atrium, and optionally through the septum into the left atrium. Driver assembly 14 may be placed on a support surface, and the driver assembly may be slid along the support surface roughly into alignment with the guide sheath 182. A proximal housing of guide sheath 182 can be releasably affixed to a catheter support of stand 72, with the support typically allowing rotation of the guide sheath prior to full affixation (such as by tightening a clamp of the support). Catheter 12 can be advanced distally through the guide sheath 182, with the user manually manipulating the catheter by grasping the catheter body and/or proximal housing 68. Note that the manipulation and advancement of the access wire, guide catheter, and catheter to this point may be performed manually so as to provide the user with the full benefit of tactile feedback and the like. As the distal end of catheter 12 extends near, to, or from a distal end of the guide sheath into the therapy area adjacent the target tissue (such as into the right or left atrium) by a desired amount, the user can manually bring the catheter interface 120 down into engagement with the driver interface 94, preferably latching the catheter to the driver through the sterile junction.

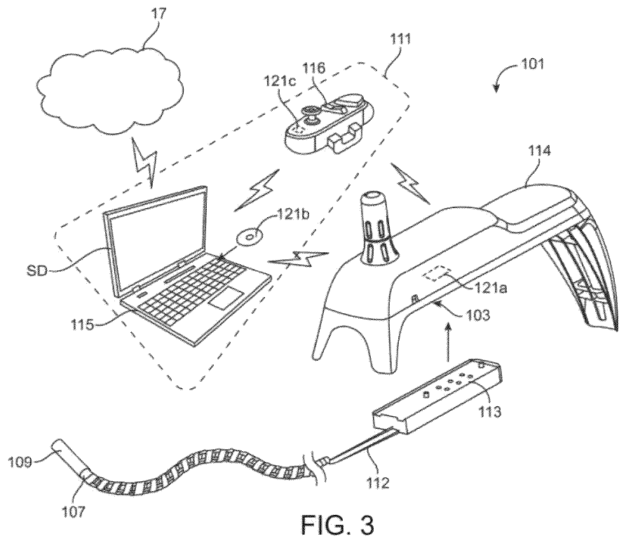

Referring now to FIG. 3, components of a networked system 101 that can be used for simulation, training, pre-treatment planning, and or treatment of a patent are schematically illustrated. Some or all of the components of system 101 may be used in addition to or instead of the clinical components of the system shown in FIG. 1. System 101 may optionally include an alternative catheter 112 and an alternative driver assembly 114, with the alternative catheter comprising a real and/or virtual catheter and the driver assembly comprising a real and/or virtual driver 114. Alternative catheter 112 can be replaceably coupled with alternative driver assembly 114. When system 101 is used for driving an actual catheter, the coupling may be performed using a quick-release engagement between an interface 113 on a proximal housing of the catheter and a catheter receptacle 103 of the driver assembly. An elongate body 105 of catheter 112 has a proximal/distal axis as described above and a distal receptacle 107 that is configured to support a therapeutic or diagnostic tool 109 such as a structural heart tool for repairing or replacing a valve of a heart. Alternative drive assembly 114 may be wireless coupled to a computer 115 and/or an input device 116. Software modules embodying machine-readable code for implementing the methods described therein will often comprise software modules, and the modules will optionally be embodied at least in-part in a non-volatile memory of alternative drive assembly 121a or an associated computer, but some or all of the simulation modules will preferably be embodied as software in non-volatile memories 121b, 121c of a computer 115 and/or input device 116, respectively.

Computer 115 preferably comprises a proprietary or off-the-shelf notebook or desktop computer that can be coupled to cloud 17, optionally via an intranet, the internet, an ethernet, or the like, typically using a wireless router or a cable coupling the simulation computer to a server. Cloud 17 will preferably provide data communication between simulation computer 115 and a remote server, with the remote server also being in communication with a processor of other computers 115 and/or one or more clinical drive assemblies 14. Computer 115 may also comprise code with a virtual 3D workspace, the workspace optionally being generated using a proprietary or commercially available 3D development engine that can also be used for developing games and the like, such as Unity™ as commercialized by Unity Technologies. Suitable off-the-shelf computers may include any of a variety of operating systems (such as Windows from Microsoft, OS from Apple, Linex, or the like), along with a variety of additional proprietary and commercially available apps and programs.

Input device 116 may comprise an off-the-shelf input device having a sensor system for measuring input commands in at least two degrees of freedom, preferably in 3 or more degrees of freedom, and in some cases 5, 6, or more degrees of freedom. Suitable off-the-shelf input devices include a mouse (optionally with a scroll wheel or the like to facilitate input in a 3rd degree of freedom), a tablet or phone having an X-Y touch screen (optionally with AR capabilities such as being compliant with ARCore from Google, ARKit from Apple, or the like to facilitate input of translation and/or rotation, a gamepad, a 3D mouse, a 3D stylus, or the like. Proprietary code may be loaded on the input device (particularly when a phone, tablet, or other device having a touchscreen is used), with such input device code presenting menu options for inputting additional commands and changing modes of operation of the simulation or clinical robotic system. As described below, movement commands may alternatively be entered by manipulating image planes of an imaging system, with the commanded position preferably being aligned with lines and/or points of intersection between the planes. Such image-based input may be used in combination with other input devices described here and/or manual movement of tools via shafts extending through robotic interventional tools, particularly when it is desirable to command movement of the robotic components without moving an image plane. For example, in a clipping procedure it may be efficient to move image planes so that a grasping image plane and a bi-commissure image plane intersect along a desired axis of the clip. Rotation of the clip about its axis so that the clip arms can open perpendicular to the line of coaptation (along the grasping plane) may be performed by manually rotating a clip delivery shaft. The user can input commands for movement of the clip along the grasping plane to effect independent grasping of each adjacent leaflet by manipulating a phone-based input device 116.

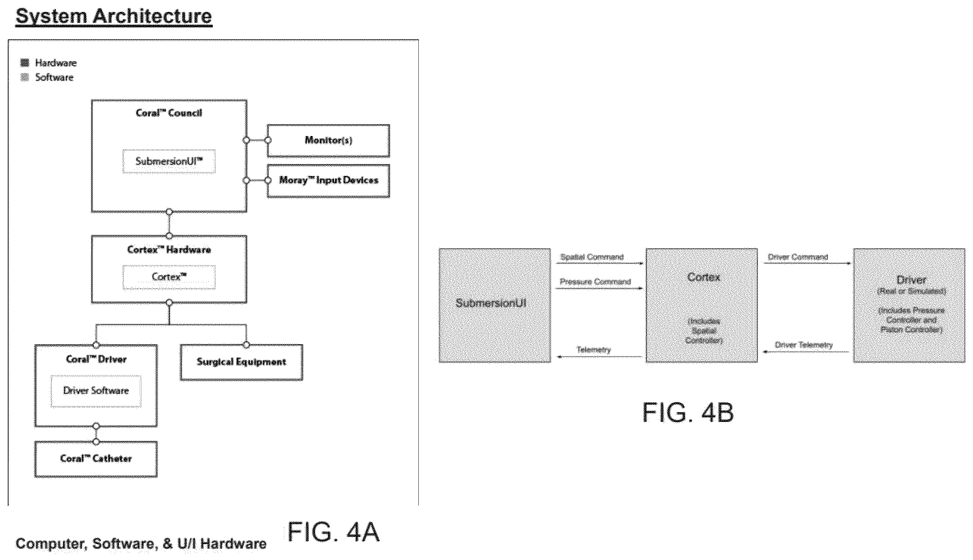

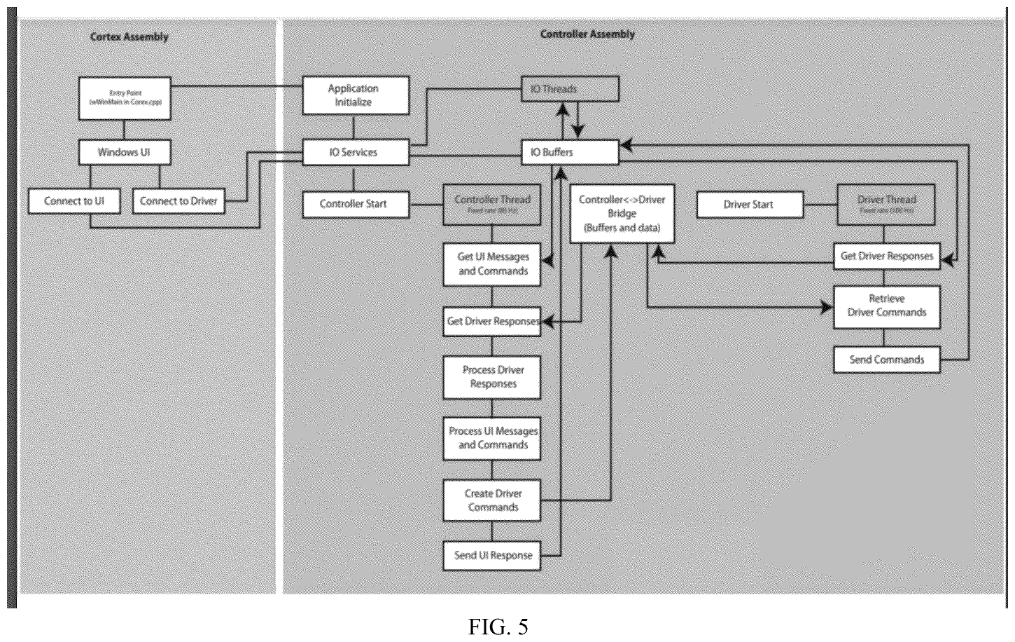

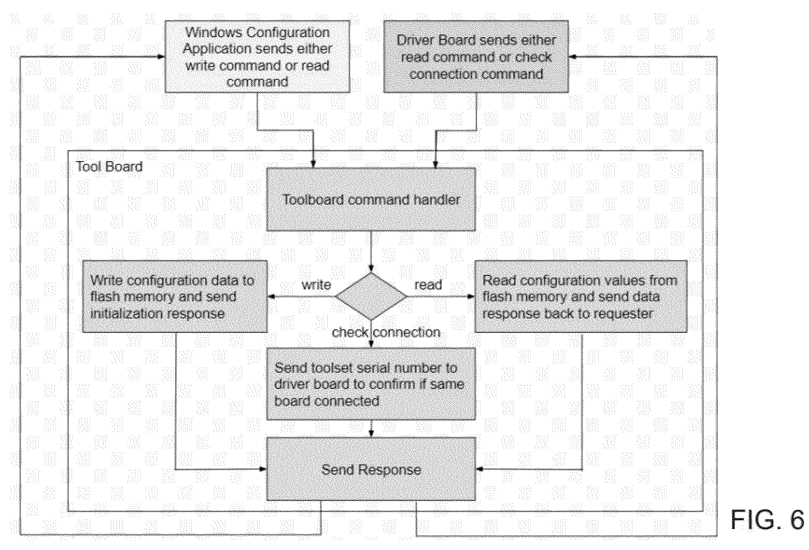

Referring now to FIGS. 4A and 4B, a data processing system architecture for a robotic catheter system employs signals transmitted between a user interface, a motion controller, and embedded circuitry of a drive assembly. As can be seen in a functional block diagram of FIG. 5, software components and data flows into and out of the motion controller of FIGS. 4A and 4B are used by the motion controller to effect movement of, for example, a distal end of the steerable sleeve toward a position and orientation that aligns with the input from the system user. The functional block diagram of FIG. 6 schematically illustrates data processing components included in each single-use replaceable catheter and data flows between those components and the data processing components of the reusable driver assembly on which the catheter is mounted. The motion controller makes use to this catheter data so that the kinematics of different catheters do not fundamentally alter the core user interaction when, for example, positioning a tool carried by a catheter within the robotic workspace.

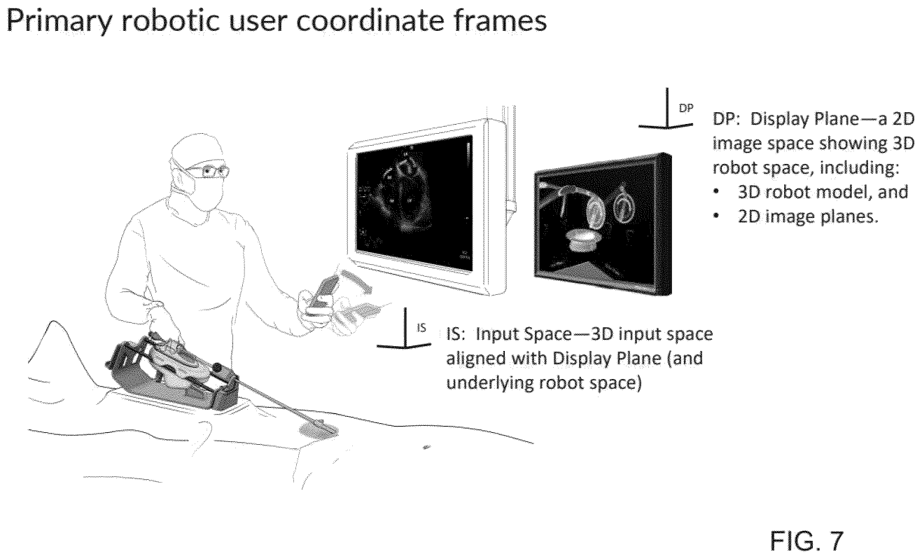

Referring now to FIG. 7, a perspective view of the robotic catheter system in the cathlab shows a clinical user of that system interacting with a 3D user input space by moving the input device therein, and with a 2D display space of the system by viewing the toolset and tissues in the images shown on the planar display. Note that in alternative embodiments the user may view a 3D display space using any of a wide variety of 3D display systems developed for virtual reality (VR), augmented reality (AR), 3D medical image display, or the like, including stereoscopic glasses with an alternating display screen, VR or AR headsets or glasses, or the like.

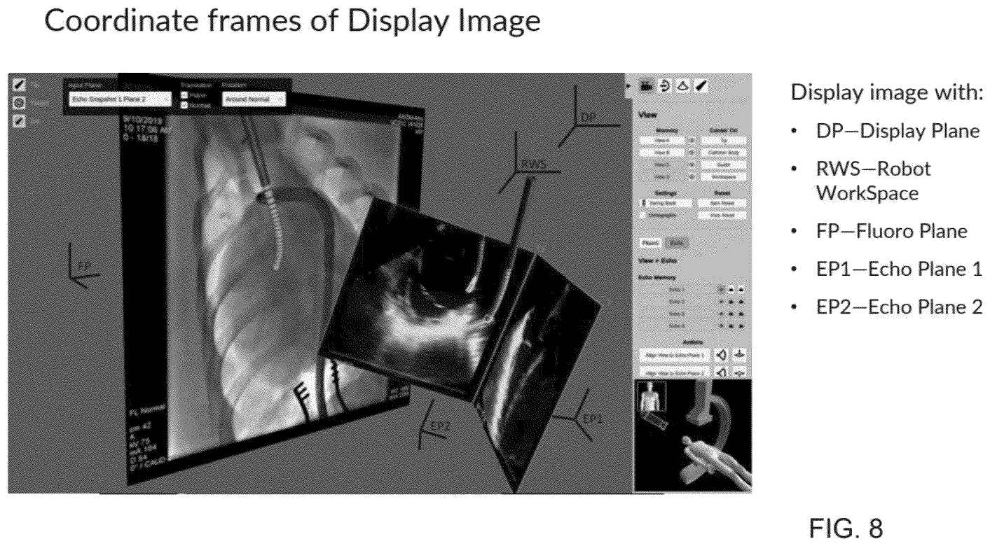

Referring now to FIG. 8, images included in the display space of FIG. 7, may include 2D and 3D image components, with these image components often representing both 3D robotic data and 2D or 3D image data. The image data will often comprise in situ images, optionally comprising live image streams or recorded video or still images from the off-the-shelf image acquisition devices used for image guided therapies, such as planar fluoroscopy images, 2D or 3D ultrasound images, and the like. The robotic data may comprise 3D models, and may be shown as 3D objects in the display and/or may be projected onto the 2D image planes of the fluoro and echo images. Regardless, appropriate positioning of the image and robotic data, as they are represented in the display space helps the user environment of the robotic system user interface to presents a coherent 3D workspace to the user. Toward that end, proper alignment of the reference frames associated with the robotic data, echo data, and fluoro data will generally be provided by the registration system of the robotic system data processor.

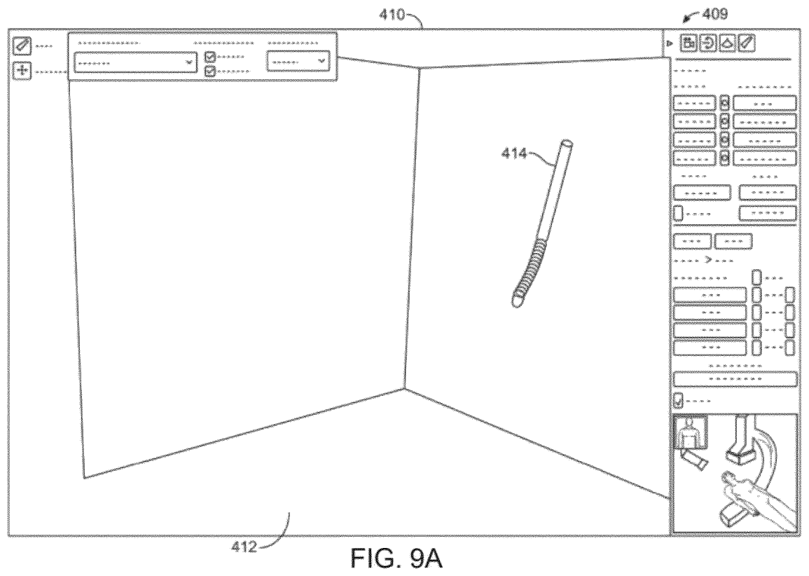

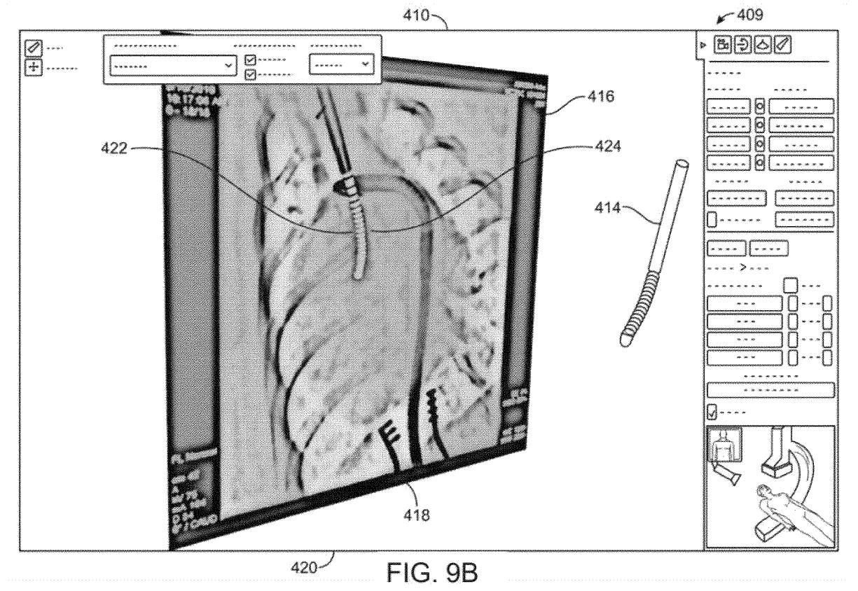

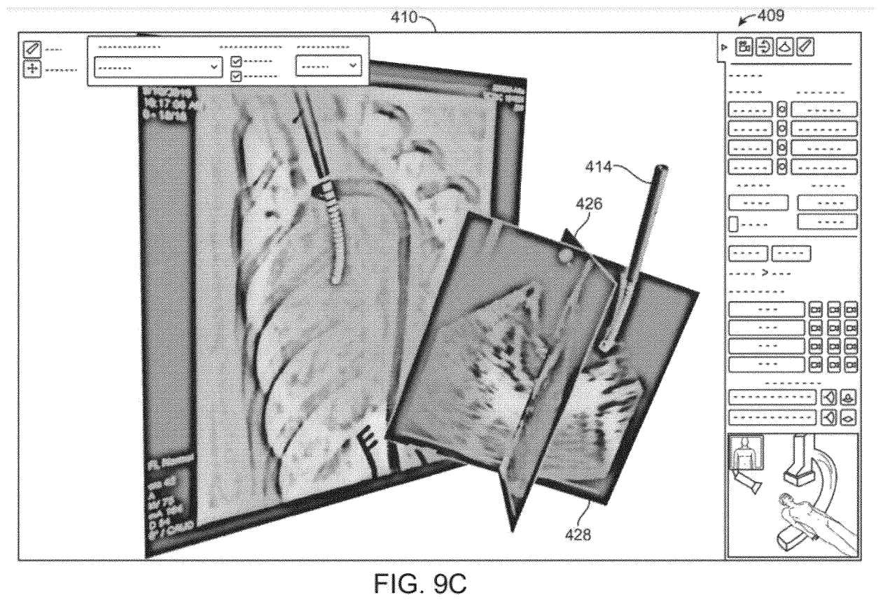

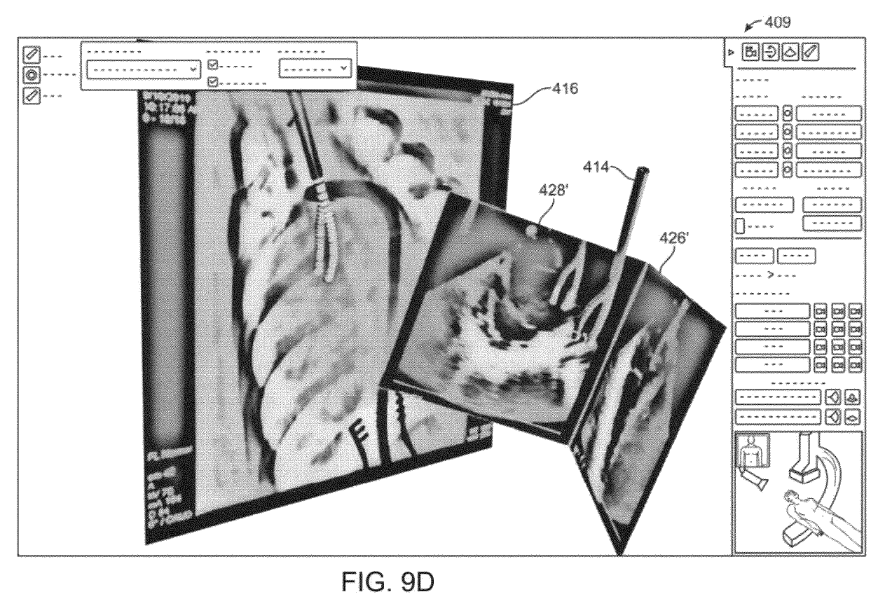

Referring now to FIGS. 9A-9D, image data from off-the-shelf or proprietary image acquisition systems and virtual robotic data may both be included in a hybrid 2D/3D image to be presented to a system user on a display 410, with the image components generally being presented in a virtual 3D workspace 412 that corresponds to an actual therapeutic workspace within a patient body. A 3D image of a catheter 414 defines a pose in workspace 412, with the shape of the catheter often being determined in response to pressure and/or other drive signals of the robotic system, and optionally in response to imaging, electromagnetic, or other sensor signals so that the catheter image corresponds to an actual shape of an actual catheter. Similarly, a position and orientation of the 3D catheter image 414 in 3D workspace 412 corresponds to an actual catheter based on drive and/or feedback signals.

Referring still to FIGS. 9A-9D, additional elements may optionally be included in image 409 such as a 2D fluoroscopic image 416, the fluoro image having an image plane 418 which may be shown at an offset angle relative to a display plane 420 of image 410 so that the fluoro image and the 3D virtual image of catheter 414 correspond in the 3D workspace. Fluoro image 416 may include an actual image 422 of an actual catheter in the patient, as well as images of adjacent tissues and structures (including surgical tools). A virtual 2D image 424 of 3D virtual catheter 414 may be projected onto the fluoro image. As seen in FIG. 9C, multiple transverse planar echo images 426, 428 may similarly be included in hybrid image 409 at the appropriate angles and locations relative to the virtual 3D catheter 414, with 2D virtual images of the virtual catheter optionally being projected thereon. However, as shown in FIG. 9D, it will often be advantageous to offset the echo image planes from the virtual catheter to generate associated offset echo images 426′, 428′ that can more easily be seen and referenced while driving the virtual or actual catheter. The planar fluoro and echo images within the hybrid image 409 will preferably comprise streaming live actual video obtained from the patient when the catheter is being driven. The virtual or actual catheter may optionally be driven along a selected image plane (often a selected echo image plane showing a target tissue) by constraining movement of the catheter to that plane, thereby helping maintain desired visibility of the catheter to echo imaging and/or alignment of the catheter in other degrees of freedom. Catheter 414, sometimes referred to as a steerable sleeve, extends through a lumen of a guide sheath 415 and distal of the distal end of the guide, with the guide typically being laterally flexible for insertion to the workspace but somewhat stiffer than the catheter so as to act as a robotic base during robotic articulation of the catheter within the workspace.

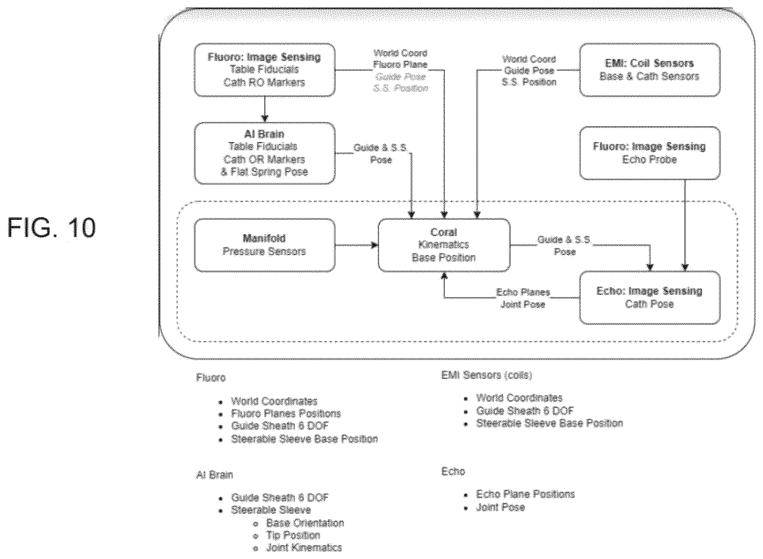

Referring now to FIG. 10, a block diagram schematically illustrating components and data transmission of a registration module generally includes a fluoro-based pose module, an echo-based pose module, and a sensor-based pose module. One, two, or three of the pose-determining modules will provide data to a robot movement control module, which may also provide robotic data regarding the pose of the articulated toolset such as pressure or fluid volume for the fluid-drive systems described herein. A fluoro image data-based pose system includes a C-arm and a fluoro image data processing module that determines an alignment between an internal therapy site and fluoro image data, and optionally a fluoro-image-based pose of one or more components of a robotic toolset in the robotic workspace, An echo image data-based system may include a TEE probe and an echo image data processing module that determines an echo-data-based pose of one or more toolset components in an echo workspace. Registration of the echo-based pose and the fluoro-based posed so as to register the echo workspace and echo image space (including the echo planes) with the robotic workspace of the 3D user environment may take place in the overall motion control module, or in a separate registration module which sends data to and receives data from the robotic motion control module. An electromagnetic (EMI) pose system (which includes an EMI sensor and a sensor data processing module) may similarly provide pose data to such an integrated or separate registration module. Note that the pose modules need not be separate. For example, data from the fluoro pose module or EMI pose module may be used by the echo pose module. Each of the pose modules may make use of data from the robotic motion control module, such as a profile or robot-data based pose of the toolset.

Referring to FIGS. 7-10, the robotic systems described herein can diagnose or treat a patient by receiving fluoroscopic image data with a data processor of the medical robotic system. The fluoroscopic image data preferably encompasses a portion of a toolset of the medical robotic system within a therapy site of the patient, with the portion typically including a distal portion of a guide sheath and/or at least part of the articulatable distal portion of the steerable sleeve. Ultrasound image data is also received with the data processor, the ultrasound image data encompassing the portion of the toolset and a target tissue of the patient within an ultrasound image field. Note that when the target tissue comprises a soft tissue such as a valve tissue of the heart, the target tissue may be more easily visible with the echo data than with the fluoro data. The data processor can determine, in response to the fluoroscopic image data, an alignment of the toolset with the therapy site, often using one or more fiducial markers that are represented in the fluoro data. The data processor can determine, in response to the ultrasound image data, an ultrasound-based pose of the toolset within the ultrasound image field. The data processor can transmit, based in-part on a desired movement of the toolset relative to the target tissue in the ultrasound image field as input by the user, and in-part on the alignment, and in-part on the ultrasound-based pose, a command for articulating the tool. To generate that command, the processor will often calculate, based on the ultrasound-based pose and the alignment, a registration of the ultrasound image field with the therapy site, with the registration optionally comprising a transformation between the ultrasound image field and the 3D robotic workspace.

Referring now to FIGS. 10-18, the fluoroscopic image acquisition system and data-based pose module may help generate the desired alignment between the reference frames associated with the fluoroscopic, echo, and robotic data. A processor of the medical robotic system can determine, in response to fluoroscopic image data, an alignment of the fluoroscopic image data with a therapy site by imaging the therapy site and a plurality of fiducial markers using the fluoroscopic image acquisition system. The fluoro system may also capture toolset image data, the toolset image data encompassing a portion of a toolset in the therapy site. The processor can calculate, in response to the captured toolset image data and the determined alignment, a pose of the toolset in the therapy site, and can use that pose to drive movement of the toolset in the therapy site using the pose.

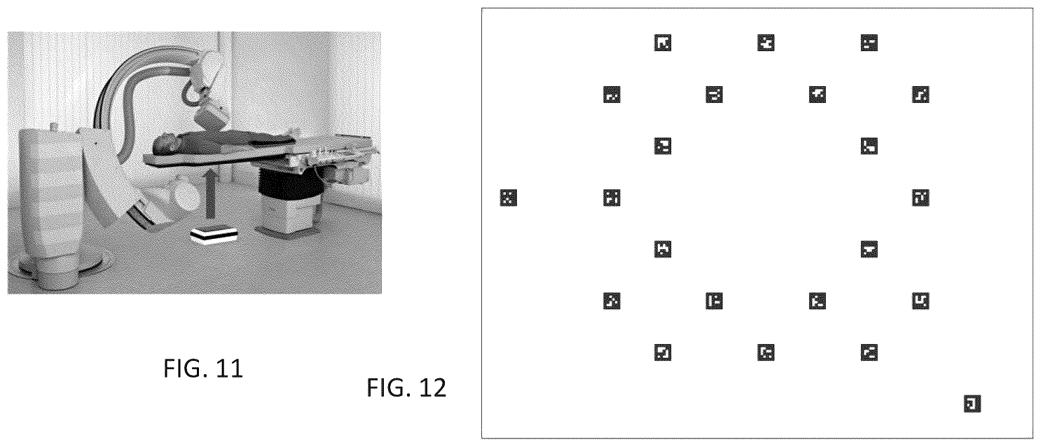

As can be seen in FIG. 11 a fluoro C-arm, CT, or cone-beam image aquation system may acquire 2D or 3D digital X-ray image data from an internal worksite within a patient. As can be understood with reference to FIGS. 11 and 12, a reference structure can be mounted to the patient support table to provide a frame of reference for the workspace. The reference structure may optionally be below the table, inset into the table, or on top of the table between the table and patient. The reference frame may comprise a substantially planar structure having a pattern of high-contrast markers, or may comprise an electromagnetic sensor system that helps localize electromagnetic tags or sensors mounted on the surgical instruments. While the reference frame associated with the reference structure is sometimes referred to as the world reference frame, it may move with movement of the table. Moreover, the tissues of the patient may move relative to the reference structure per cyclical physiological movement or gross movement of the patient. Nonetheless, the reference frame may be used to generate pose data of the workspace, tissues, and instruments therein, with or without adjustment for such patient movement.

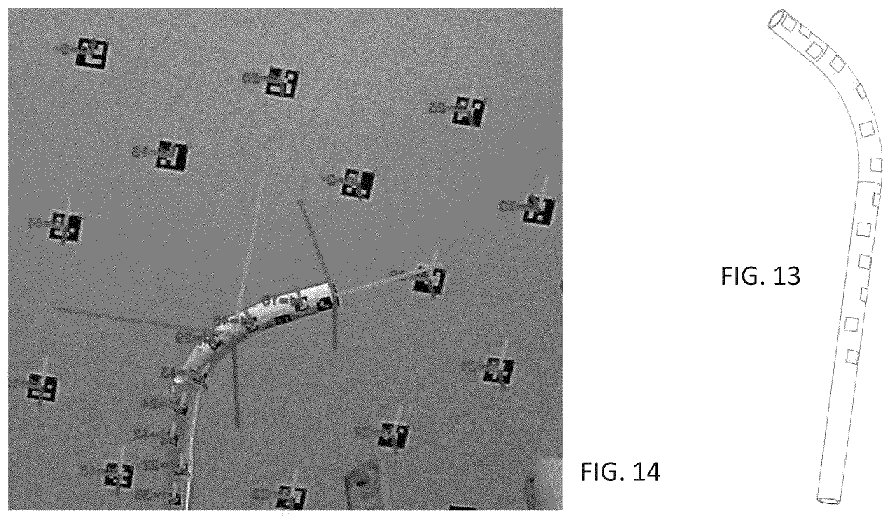

Referring now to FIGS. 12-14, patterns of markers may be included on the reference structure and at least some of the tools introduced into the patient to facilitate determining of world, image plane, and tool poses in the workspace using image processing of the guidance images. For the X-ray reference structure shown in FIG. 12 and guide sheath shown in FIG. 13 (both shown together in an optical image FIG. 14), high-contrast fiducial markers for fluoro imaging may be laser cut from tantalum. Automatic recognition and localization of the markers may be facilitated by the use of markers embodying machine-readable 1D or 2D codes that have been developed for camera-imaging, such as Aruco markers or the like. These fiducial markers may be included in a fiducial marker plate such as that shown in FIG. 12. The fluoro-based pose module code for processing fluoro image including some or all of the markers from such a plate may again use the OpenCV software library.

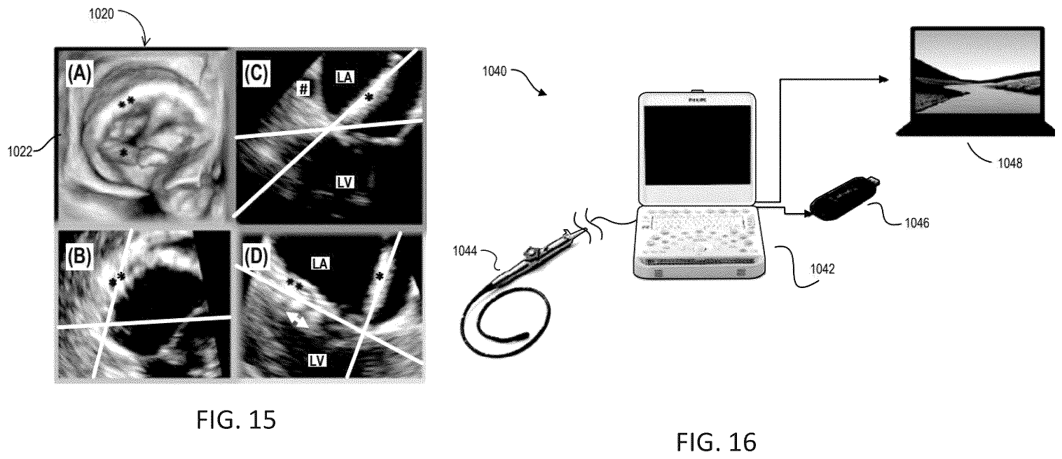

Referring now to FIG. 15, an alternative echo display 1020 includes both 3D image elements 1022 and 3 planar echo images. Such multi-planar reconstruction (MPR) echo display arrangements may be registered and incorporated into a single hybrid 2D-3D display by determining the poses for each of the planar echo images and adding those planar images to the hybrid display using the multi-thread registration system described hereinbelow. The 3D image element may similarly be registered and its 2D representation (as shown in the MPR display) added in the area of the hybrid display showing the 3D model of the toolset with the 3D toolset model superimposed thereon. More details on this MPR display may be seen in an article entitled: Feasibility of a MPR-based 3DTEE guidance protocol for transcatheter direct mitral valve annuloplasty; Martin Geyer MD, et. al., 10 Aug. 2020 https://doi.org/10.1111/echo.14694.

Referring to FIG. 16, an exemplary TEE system 1040 for use in the technologies described herein comprises an ultrasound machine 1042 such as a compact Philips CX50 and associated TEE probe 1044 such as an X7-2T probe. The data captured by the TEE probe can be exported in processing in at least two ways: by direct upload of DICOM data format (via USB transfer), or by live streaming of data from the ultrasound machine in MP4 format via an external capture card 1046 (screen capture), such as with an Elgato Cam Link 4K camera capture card. Alternative capture cards incorporated into a data processor of a computer 1048 running some or all of the image processing code described herein could also be used. Live streaming of the data (MP4 format via external capture card) may be preferred over standard DICOM data due to a simplified workflow (no manual upload) and greater accessibility to meta data, though in some cases additional non-standard DICOM image streaming with meta-data regarding the ultrasound acquisition settings and the like may have more advantages.

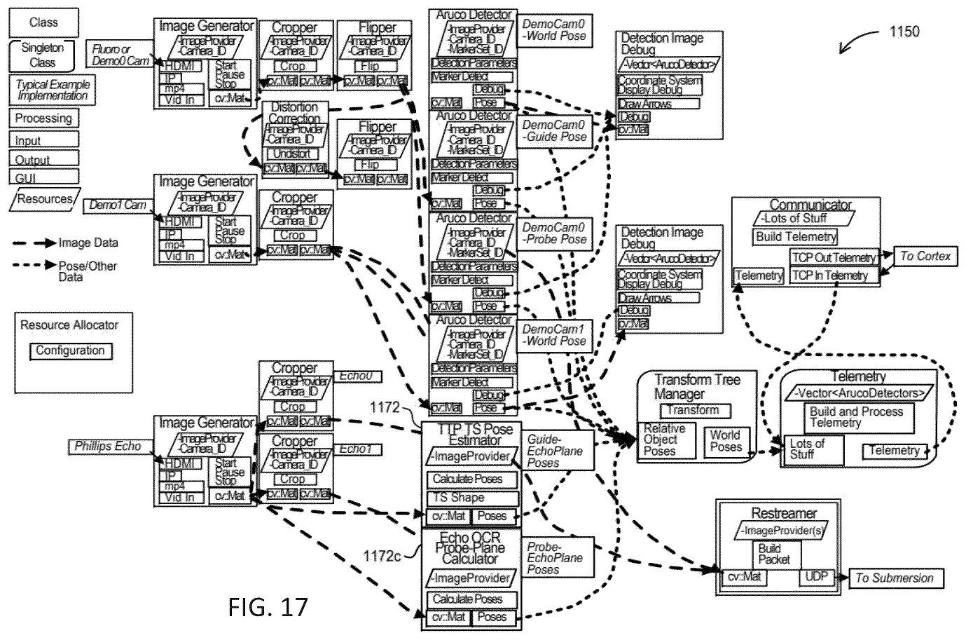

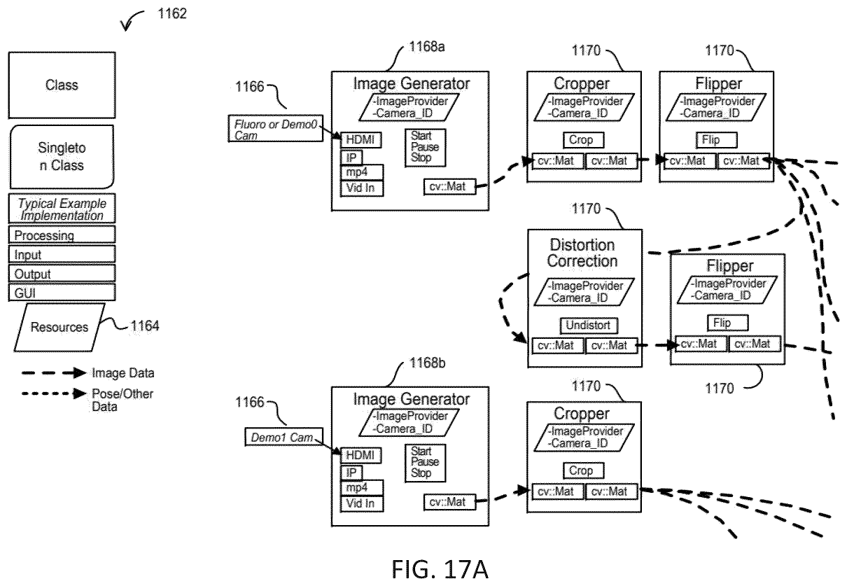

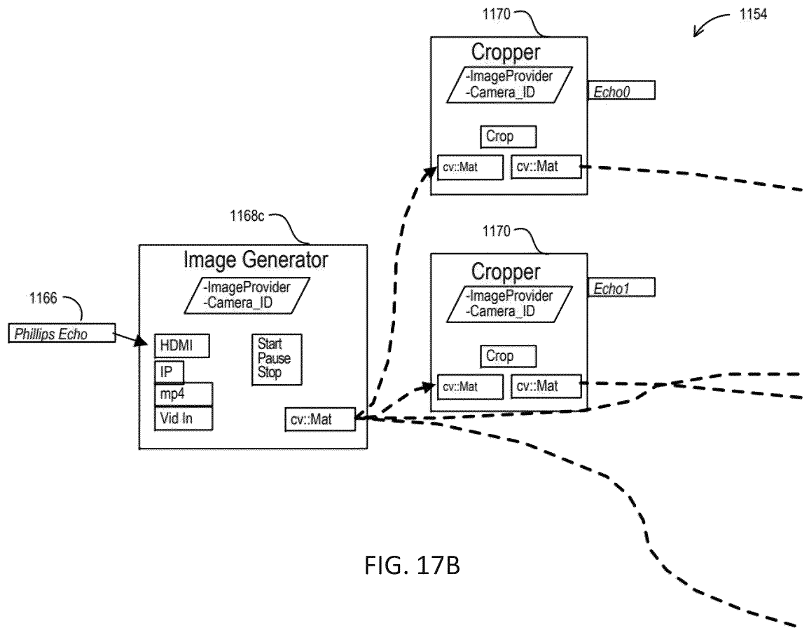

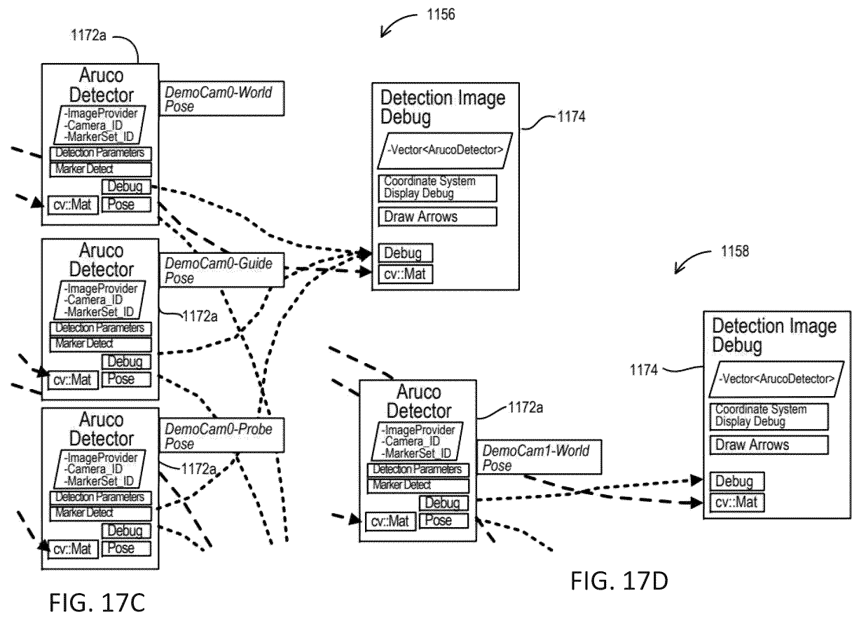

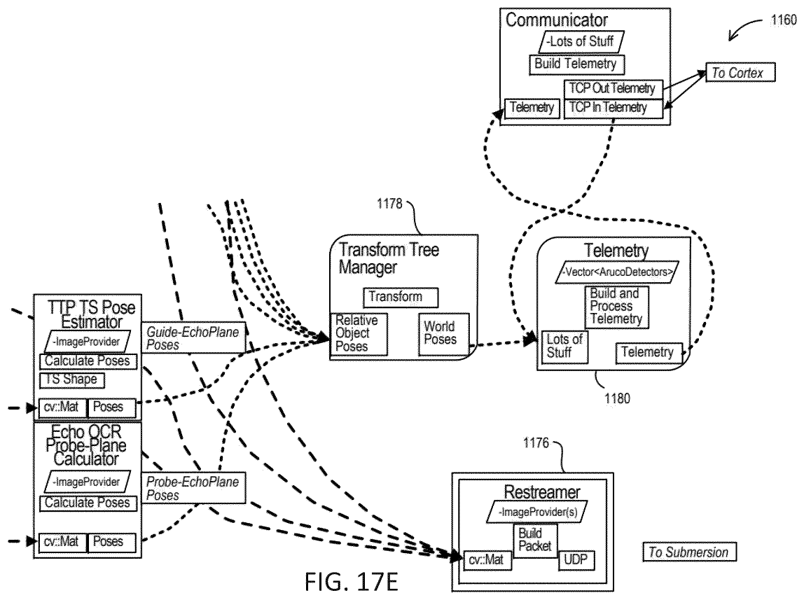

Referring now to FIGS. 17-17E, an exemplary multi-thread registration module 1150 can accept image input from multiple image sources (including an echo image source as well as one or more additional image sources operating on different remote image modalities). In response to the image data streams, the various components of the registration module provide pose data of echo image planes acquired by the echo image source, pose data of multiple interventional components of an interventional toolset shown in those echo image planes, and modified image data streams. For convenience the components of the registration module may be referred to as sub-modules, or as modules themselves. The complete module is illustrated in FIG. 17; different portions 1152, 1154, 1158, and 1160 of registration module 1150 are shown in FIGS. 17A-17E in larger scale, and a legend 1162 is included in FIG. 17A. Note that additional resources 1164 may also be included in the overall registration module package, with two of the references of note being transformation manager data and a listing identifying the various sub-modules and data pipelines between these sub-modules of registration module 1150. The transformation manager data will include a listing of all the reference frames used throughout the overall registration module, starting with a root or patient frame (sometimes called the “world” or “robot” space). The root space is optionally defined by the pose of the fiducial marker board supported by the operating table. All other reference frames can be dependent on or “children of” (directly or indirectly) the root space so that they move when the root space moves (such as when the operating table moves). All of the parent/child relationships between the reference frames are specified in a transformation tress of the transformation manager data.

Referring now to FIGS. 17A and 17B, a series of image stream inputs 1166 receive data streams from image sources (such as an ultrasound or echo image acquisition device, a camera, a fluoroscope, or the like) for use by a series of image generators or formators 1168, including a fluoro image generator 1168a, a camera image generator 1168b, and an echo image generator 1168c. The image generators are configured to format the image stream for subsequent use, such as by converting an MP4 or other image stream format to an OpenCV Image Datastream format. Note that multiple different image generators may receive and format the same input image stream data to format or generate image data streams for different pose tasks. Regardless, the formatted image data streams can then be transmitted from the image generators 1160 to one or more image stream modifiers 1170. There are a variety of different types of image stream modifiers, including croppers (which crop or limit the image area of the input image stream to a sub-region), flippers (which flip the image data left-right, up-down, or both), distortion correctors (which apply correction functions to the pixels of the image stream to reduce distortions), and the like. A series of image stream modifiers may be used on a single image stream to, for example, crop, flip, remove distortions, and again flip the fluoro image stream data. The image modifiers may transmit modified image streams to different sub-modules, optionally applying different modifications. For example, a cropper may crop multiple regions of interest from the echo image stream, sending the primary plane to one sub-module and a region of an acquisition parameter to an optical character reader sub-module. Alternatively, the cropping of separate regions of a particular input image data stream may be considered to be performed by separate croppers, as in both cases the actual code may involve running different instances of the same cropping sub-module. The output from these modification modules generally remains image data streams (albeit modified).

Referring now to FIGS. 17C, 17D, and 17E, the modified image data streams from the image modifiers are transmitted to a number of different image analyzers and augmentors 1172. These sub-modules analyze the image stream data to generate pose data and/or to augment the image streams, most often superimposing indicia of pose data on the image streams. First addressing a sub-group of the image analyzers identified as Aruco detectors 1172a, these sub-modules detect specific markers (Aruco or others) in the modified image streams they receive, and in response, transmit pose data determined from those markers. The Aruco detectors may identify only a specific set of Aruco or other markers which the registration resources list for that sub-module. Once the markers are identified, pixels at corners (or other key points) of the identified markers may determine in the 2D image plane, and the determined corners from all the identified markers may be fed into a solver having a 3D model of the interventional component. The 3D model has those corners, allowing the solver to determine a pose of the interventional component in the image stream. Aruco detectors 1172 may also transmit an image stream that has been augmented by a marker identifier/localizer, such as an outline of the marker, a small reference frame showing a center or corner of the marker and its orientation, or the like.

Referring still to FIGS. 17-17E, another sub-group of the image analyzers comprises an echo-based toolset pose estimator (which may use echo sweeps to generate 3D point clouds and derive toolset pose data in echo space as described above). A final sub-group of the image analyzers comprises an echo probe plane pose calculator 1172c which determines a location of an echo probe (TEE or ICE), and also the poses of the image planes associated with that probe (often using OCR data). Once again, these analyzers generate both pose data and augmented image data streams with indicia of the pose data.

Referring to FIGS. 17D and 17E, the modified and/or augmented images may be transmitted to debug modules 1174 for review and development, and/or to a re-streamer 1176 that publishes the image data streams for use by the GUI module for presentation to the user (as desired), or for use by other modules of the robotic system. The pose data from all the image analyzers are transmitted to a transformation tree manager 1178 that performs transforms on the pose data per the transformation manager data (described above) so as bring some or all of the pose data into the root space (or another common frame). That pose data can then be packaged per appropriate protocols for use by other modules in a telemetry module 1180 and transmitted by a communication sub-module 1182.

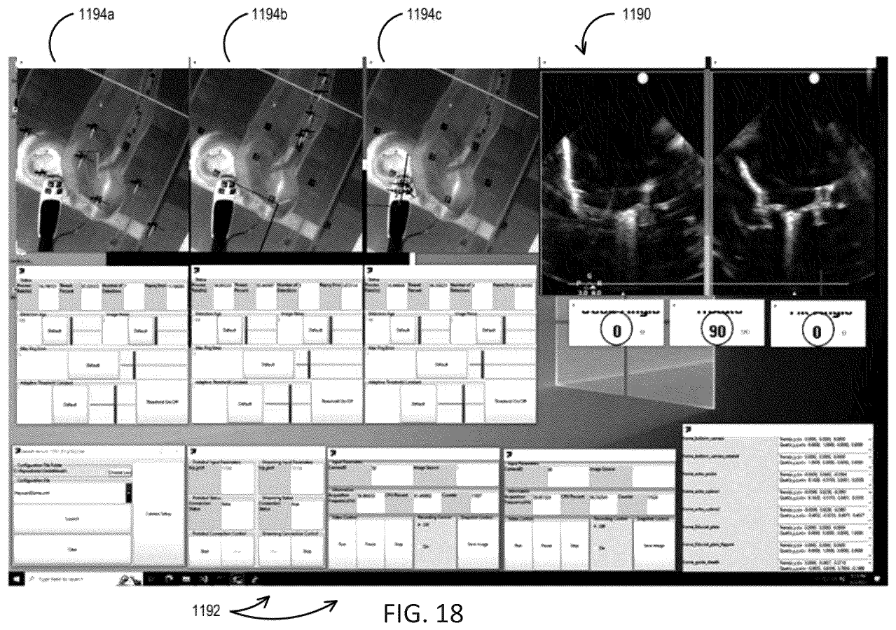

Referring now to FIG. 18, a registration module GUI 1190 includes a series of windows 1192 associated with selected sub-modules, along with a series of image displays. The sub-module windows present data on pose error projections and the like, and allow the user to vary pose generation parameters such as by introducing and varying image noise (which can be beneficial), varying pose error projection thresholds (such that low-confidence pose data may be excluded), varying thresholding parameters for image processing, or the like. The image displays may comprise augmented image displays showing, for example, a marker board image display 1194a showing identified reference frames for individual markers and the overall marker board as identified by a “world pose” Aruco detector module, a guide sheath display 1194b showing the identified frames of the individual guide sheath markers and an overall guide sheath frame, a TEE probe display 1194c showing the identified frames of the TEE probe, and so on.

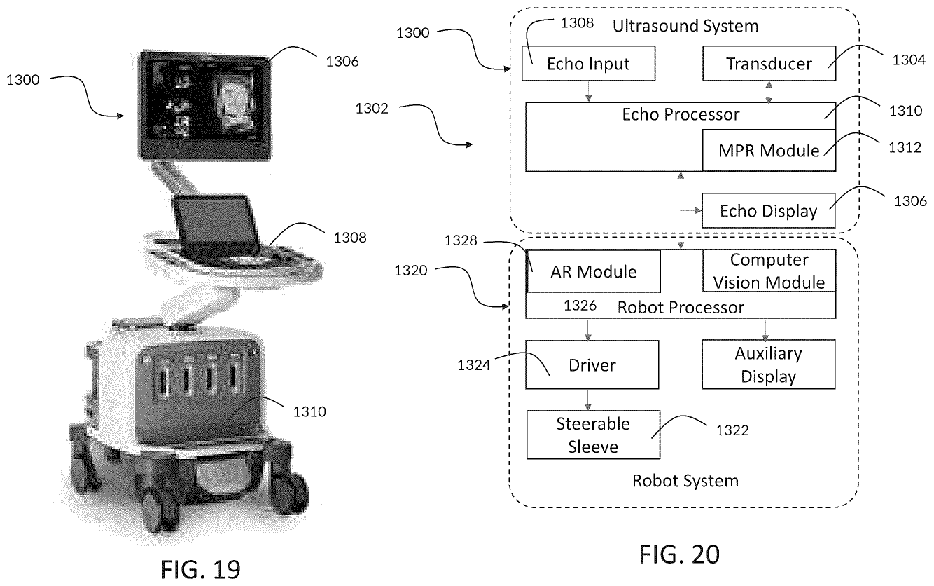

Referring now to FIGS. 19 and 20, the structure and use of a combined image and robotic system 1302 with an MPR-capable ultrasound system 1300 can be understood. Ultrasound system 1300 includes a transducer for generating three-dimensional (“3D”) image data of the 3D workspace within the patient data. The transducer will typically be included in a TEE or ICE probe. The ultrasound system also includes a display 1306 for showing a plurality of planar images of the 3D workspace and an input 1308, typically in the form of a trackball though a touchscreen, keyboard, mouse, and/or other input system may be provided. A processor 1310 of the ultrasound system will often include a multi-plane reconstruction (“MPR”) module 1312 coupling the transducer and the input to the display so that the planar images on the display show the 3D data adjacent associated imaging planes, and so as to facilitate manipulation of the imaging planes using the input. As will be described in more detail hereinbelow, an intersection of the imaging planes may be used to define an image-based position and orientation within the 3D workspace.

Referring still to FIGS. 19 and 20, the combined system 1302 also includes a robot system 1320. The robot system has an articulating arm 1322 and a proximal driver 1324. The articulating arm will often comprise an articulating catheter structure having a proximal end and a distal end with an axis therebetween as described above. The proximal end of the articulating arm is couplable with the driver, and the driver may induce bending of the axis of the arm using a fluid drive system, using pullwires, using concentric bent tubes or other continuum robotic structures, using linkage systems having a series of joints between rigid links, or the like. The distal end of the articulating arm will typically be configured for insertion into the 3D workspace as described above. The driver can be coupled with the processor of the ultrasound system, typically by a processor of the robotic system 1326. Alternatively, the robotic and ultrasound processor circuitry can be integrated or separated into any of a wide variety of alternative distributed data processing architectures. Regardless, the coupling of the driver to the ultrasound system will often be configures so as to induce driving of the distal end of the articulating arm toward the image-based position and orientation (as defined by the user manipulating the input of the ultrasound system). Note that this can allow the robot to be driven at least in part using the graphical user interface of the ultrasound system.

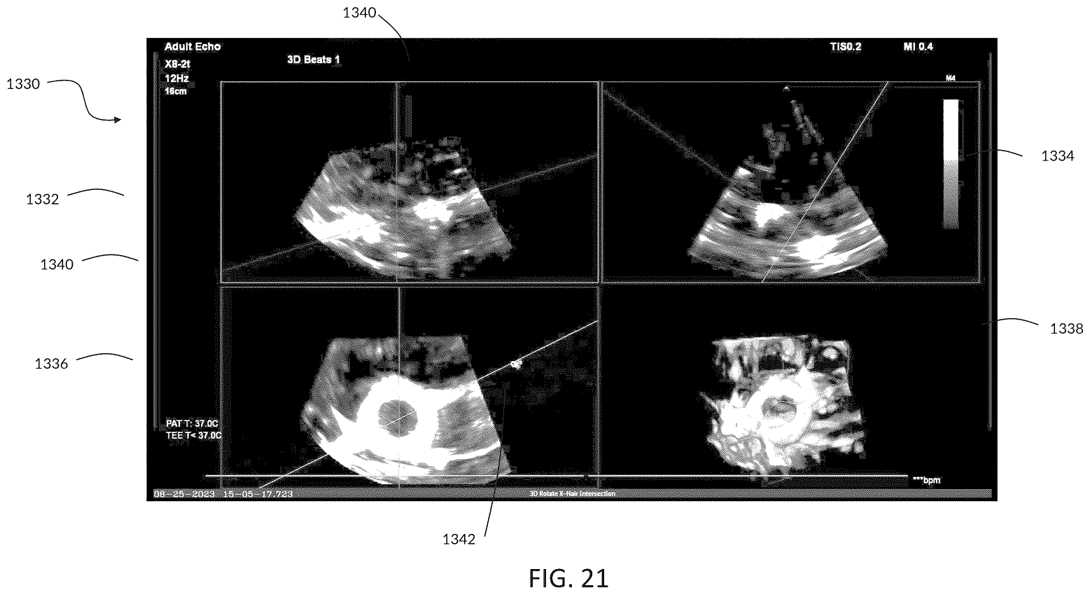

Operation of the graphical user interface of ultrasound system 1300 to manipulate the image planes within the workspace and set the desired tool position and orientation can be understood with reference to FIGS. 19 and 21-23B. As shown in FIG. 21, a screen print 1330 of the echo display may optionally present a series of planar images 1332, 1334, and 1336, along with a 3D image 1338. The green, red, and blue planar images 1332, 1334, and 1336 each have associated image planes within the workspace and are presented in windows outlined by associated colors, for example, planar image 1332 is outlined in green and has an associated image plane shown in green in the other windows, planar image 1334 is outlined in red and has an associated image plane in the workspace that is shown in red in the other windows, and planar image 1336 is outlined by a blue box and has an associated plane that is blue in the other boxes. Within each box, the image planes associated with the other two windows are illustrated by associated lines 1340. For example, within the green planar image 1332, the image plane associated with the red planar image 1334 is represented by a red line, and the image plane associated with the blue planar image 1336 is represented by a blue line, with these lines showing the intersection between the green image plane and the red image plane, and between the green image plane and the blue mage plane, respectively. The other plane intersections are similarly represented. To manipulate the planes within the workspace, the clinician can move an icon (such as hand icon 1342) to an end portion of the green line 1340 within the blue planar image 1336 using the trackball input, engage a button, and move the hand icon to change the angle of the line and associated plane. Moving the hand icon to a central portion of the line translates the line and associated plane laterally relative to the line. Alternative image plane manipulation inputs may also be available, with the ultrasound system generally allowing the user to set the position and orientation (together, the pose) of the image planes within the workspace. The 2D planar images shown in each window are generated or reconstructed from 3D echo data sensed by the transducer using a portion of the 3D data adjacent the associated image plane.

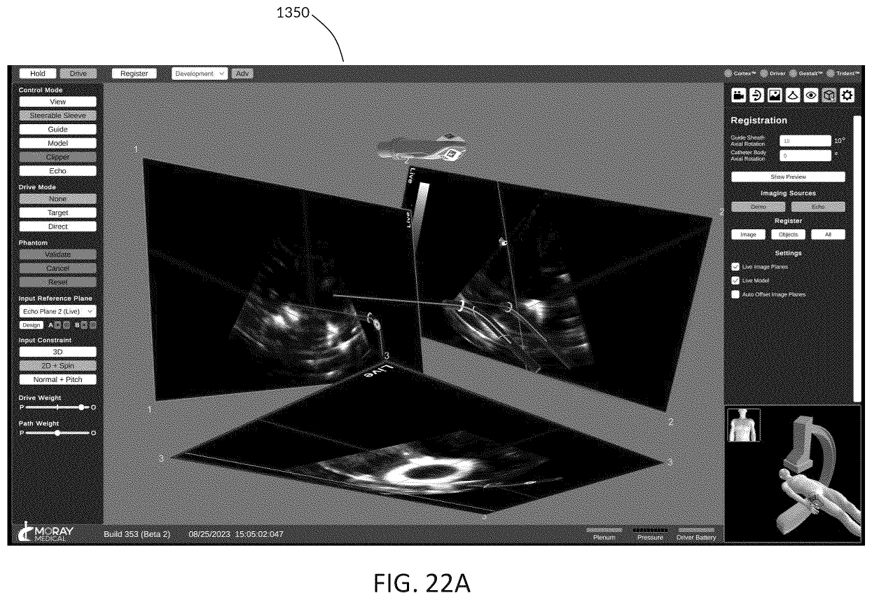

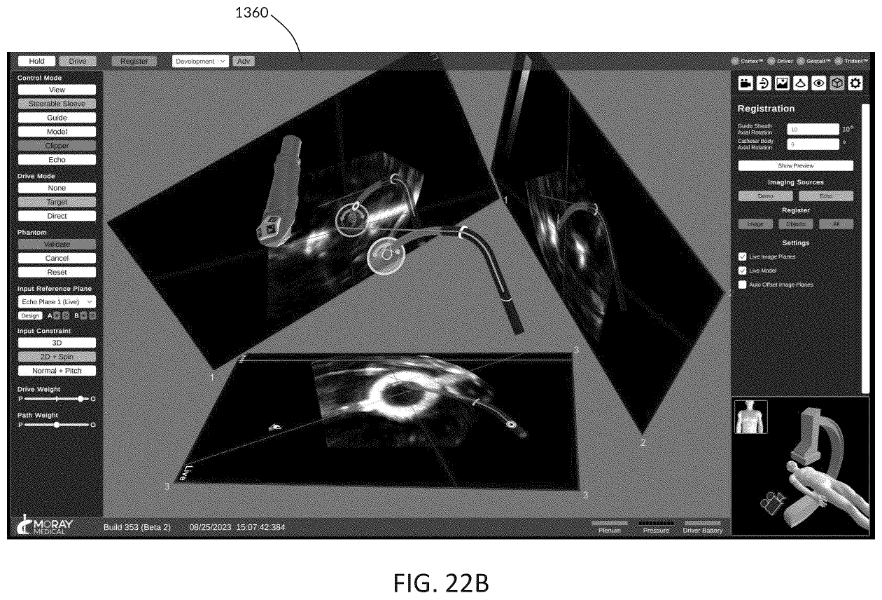

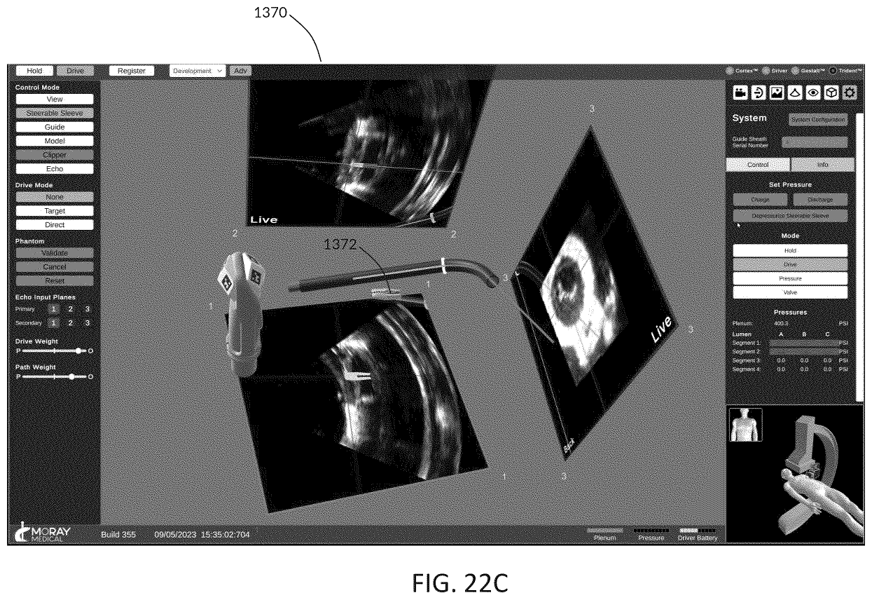

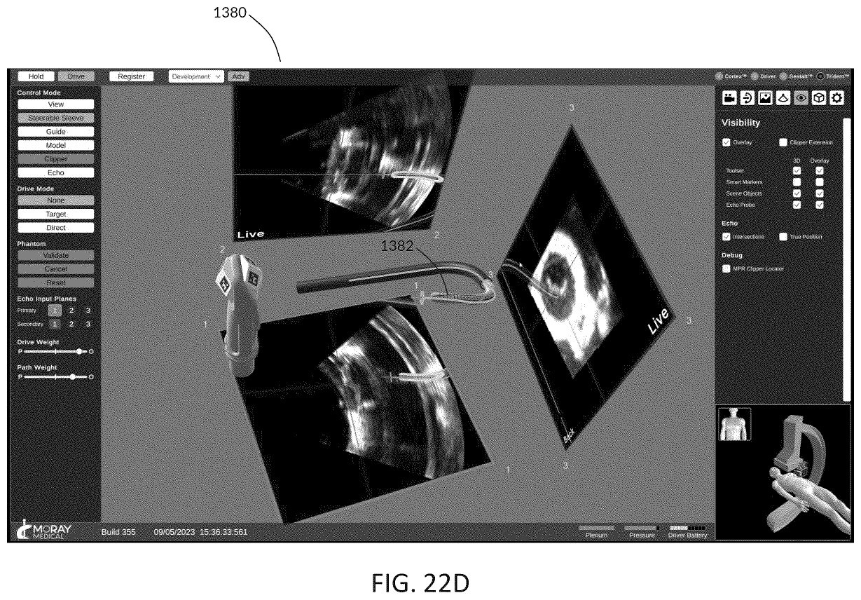

As can be understood with reference to FIGS. 22A and 22C, the combined image and robot system can take advantage of the manipulation of the echo image planes for a different purpose: establishing a target pose for the distal end of the robot arm and any therapeutic and diagnostic tool supported thereon. As can be understood from the description above and from FIG. 22A, a screen print 1350 from the hybrid 2D/3D auxiliary display of the robot system shows a 3D workspace generated using a combination of 2D, 3D, virtual, and actual image data. The 3D poses of the 2D green, red, and blue planar echo images are based on the 3D poses of the green, red, and blue echo imaging planes within the workspace in the patient body, with the planes offset from their intersections as described above. 3D AR images of the articulated catheter, guide, and ultrasound probe are generated from 3D models of these structures and superimposed in the virtual workspace with poses corresponding to the actual articulated catheter, guide, and ultrasound probe in the actual workspace, as sensed using image processing, electromagnetic sensor systems, or the like.

Referring now to FIGS. 22A, 22B, and 22C when preparing to move an interventional tool to a target tissue, the image planes of the ultrasound system will often be moved into alignment with that target tissue. For example, as shown in the screen print 1360 of the auxiliary display in FIG. 22A, the blue image plane has been positioned roughly coplanar with a model valve annulus. The red image plane may traverse the valve leaflets in a grasping view, while the green plane may, for example, may provide a bi-commissural view extending along the line of coaptation of the leaflets. This positions the intersection of the green and red image planes along a desirable trajectory for a TEER clip axis, with the intersection of all three planes defining an intersection point adjacent a center of the valve. As shown in FIG. 22B, the clinician can then steer a virtual delivery catheter in the 3D workspace (optionally sequentially along the image planes) and into a desired alignment with the target tissue. Alternatively, when a TEER clip or other therapeutic or diagnostic tool is supported by the distal end of the catheter, an augmented reality (AR) module 1328 of the robotic processor (see FIG. 20) can help couple the processor of the imaging system with the auxiliary display and can superimpose an image of a virtual therapeutic or diagnostic tool 1372 on the auxiliary display, as seen in the screen print 1370 of FIG. 22C, with the axis of the clip extending along the red-green intersection axis, and with the distal end of the clip at the intersection of all three planes. Hence, the orientation and position of tool 1372 can be based at least in part on the poses of the image planes. More specifically, the ultrasound system generates a data stream during use, which a processor of the robot system can use to determine a pose of the transducer in the 3D workspace (optionally using either image processing). An image processing module that couples the processor of the ultrasound system with the driver can identify poses of the image planes relative to the transducer in response to the image data stream, or the image plane pose data may be provided from the processor of the ultrasound system. The processor of the robot system can transmit tool movement commands to the driver so as to induce movement of the distal end of the robotic catheter in response to the pose of the transducer and the poses of the image planes during use.

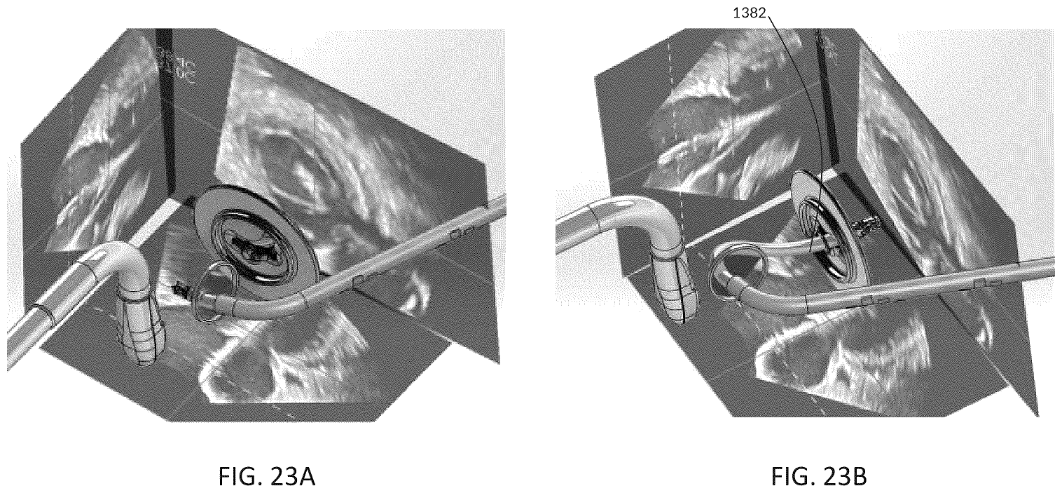

Referring now to FIGS. 22C, 22D, 23A, and 23B, along with establishing a target pose of the tool 1372 or distal end of the robotic catheter (such as that based at least in part on the positioning of the image planes in the workspace), an actual or virtual guide sheath pose in the workspace may also be set, such as by manually introducing the guide sheath with markers thereon and measuring a guide sheath pose using image processing, or by manipulating a virtual guide sheath using image planes, a phone-based input device, or other robotic input devices. The robot processor can then determine a shape 1382 of the robotic catheter (often defined by one or more bends in an axis of the catheter, as described above) which, when the catheter is advanced through the guide sheath with the tool thereon, will support the tool in the desired pose. If the workspace of the robotic catheter does not encompass the desired tool pose, an indication may be provided to the clinician to facilitate repositioning of the actual or virtual guide sheath. Optionally, the clinician may move one or more points located along the length of the actual or virtual catheter body along a selected image plane to alter the axial curvature of the catheter body, such as to avoid impinging adjacent tissue (for example, when encountering an “aorta hugger” situation in a mitral clipping procedure). Still other alternatives may also be employed, including allowing the clinician to identify a series of points or draw a curve along one or more of the image planes, with the processor fitting an axial curvature of the robotic catheter to or near the desired shape. Note that the transducer used to generate the 3D image data may optionally comprise an intracardiac echocardiography (“ICE”) transducer supported by the robotic catheter, thereby providing robotic ICE transducer manipulation. Regardless, once the AR module has been used to display an AR catheter body extending from a guide sheath to the tool, and after any desired trajectory modification input has been entered for altering an AR axis of the AR catheter body, an insertion control module of the robotic processor can be used to varying a shape of the catheter body axis so as to induce following of the AR axis by the distal end during axial advancement of the catheter toward the target tissue.

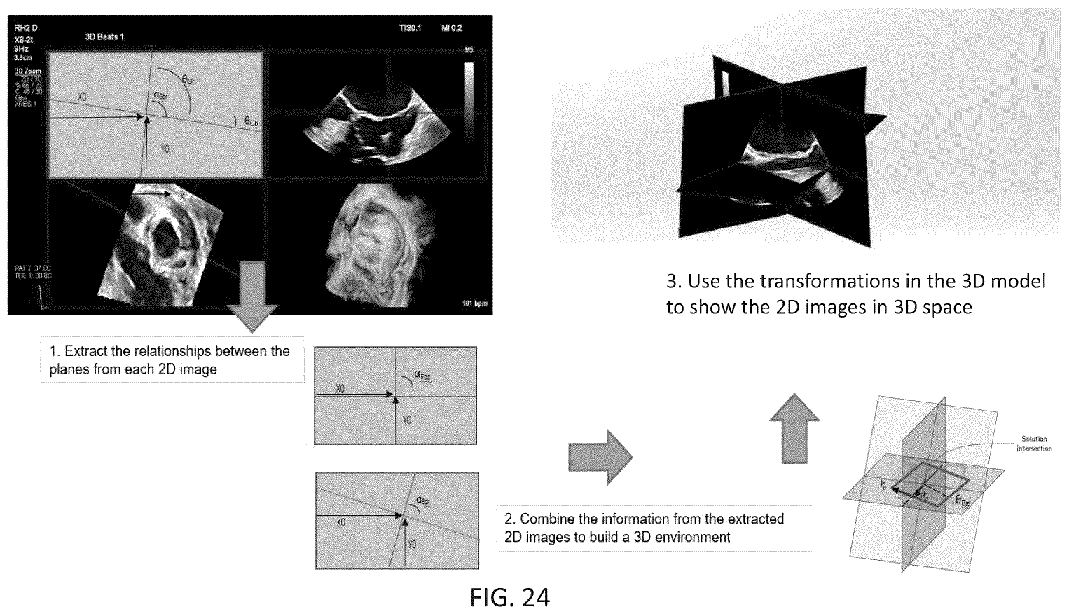

Referring now to FIG. 24, steps that can be employed by a computer vision or image processing module of the robotic processor to determine poses of the image planes relative to the ultrasound transducer from an echo image data stream are introduced. First, the relationships between the planes are extracted from the planar echo images included in the image stream, and more specifically from the lines on each planar images that identify the planar intersections. Second, this plan intersection information is then used to assemble a 3D environment, and to infer the poses of each of the planes relative to the transducer. The pose data of the planes and transducer can then be transmitted to a multi-thread registration system, allowing the generation of transformations which help show streaming 2D images in the hybrid 2D/3D robotic workspace.

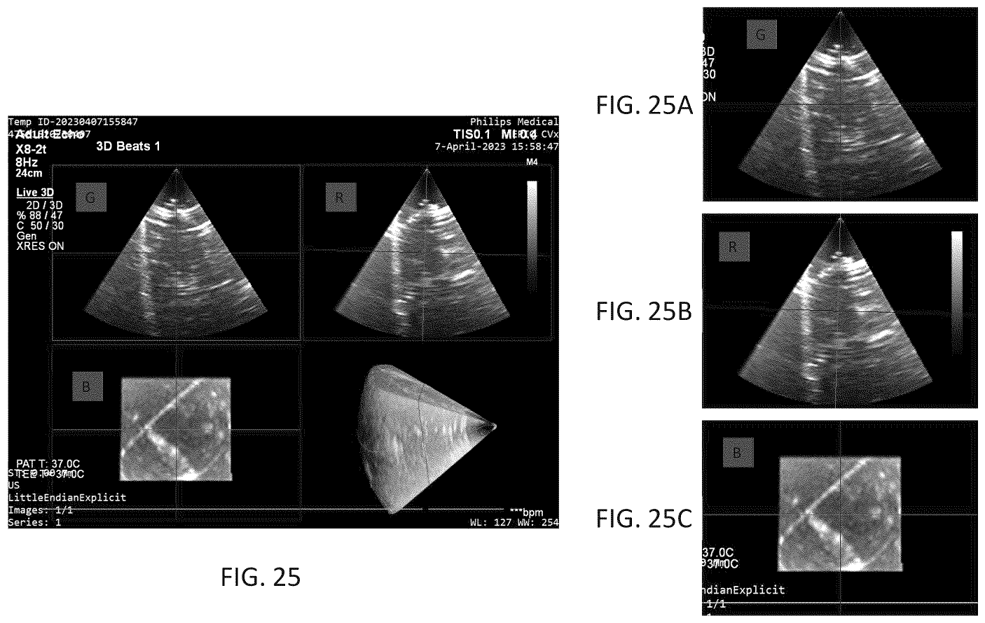

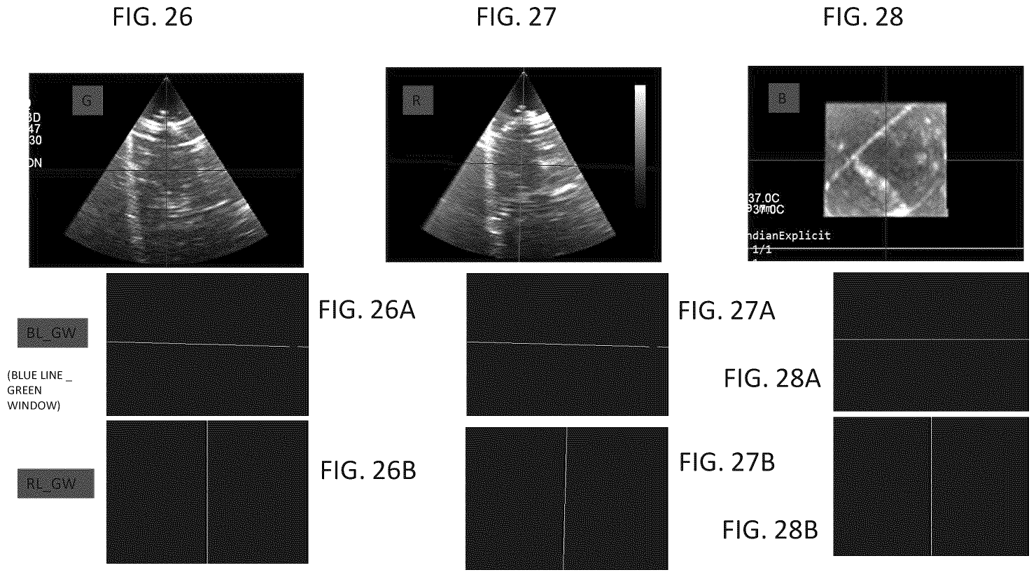

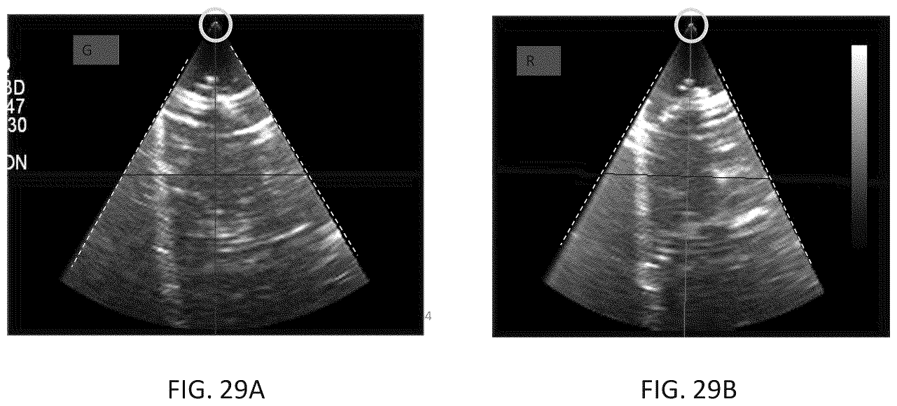

Reviewing the image processing steps of FIG. 24 in more detail and starting with FIGS. 25-25C, the windows of the individual green, red, and blue planar images are cropped from the overall ultrasound display of FIG. 25 to provide a separated green planar image of FIG. 25A, a red planar image of FIG. 25B, and a blue planar image of FIG. 25C. As shown in FIGS. 26-28B, red and blue planar intersection lines can be identified from the green planar image, green and blue planar intersection lines can be identified from the red planar image, and red and green planar intersection lines can be identified from the blue planar image, all using color mask techniques available in OpenCV and other image processing libraries. As can be understood with reference to FIGS. 29A and 29B, a tip of the echo sensing cone may then be determined using two of the cropped planar images. More specifically, the image processing module can run a color mask on the images to find any white/gray pixels that define an echo cone. Other known items in the image that are not the echo data (e.g., text, color scale bar) may be removed. A computer vision (“CV”) library and image manipulation techniques can be used to find the shape of the Echo Cone, and edge detection can be used to find the lines that make up the sides of the Echo Cone. The point of the “Tip” of the Echo Cone in the image can then be identified. It may be advantageous to determine the Echo Cone and Tip position while the image planes are in their default or home position before user manipulation of the planes, optionally while contrast is adjusted to more clearly differentiate the Echo Cone. Based on transducer calibration measurements and/or assumptions regarding the initial probe location in that home position, and on the location of the tip of the Echo Cone in the Green Image, the transform relationship between the Green Image the probe pose may be set.

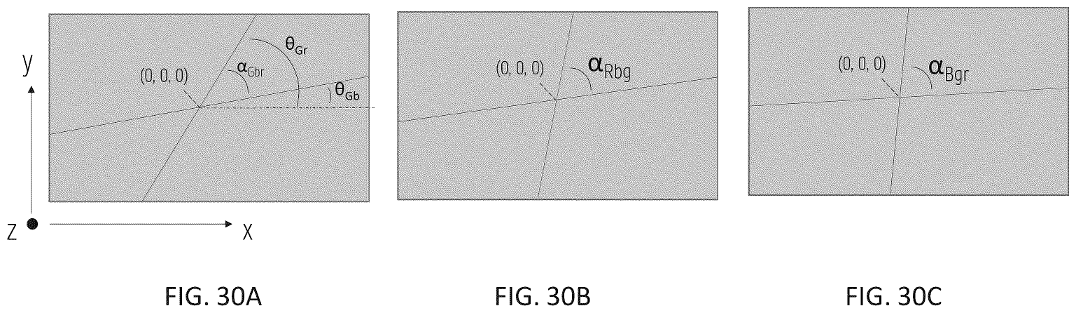

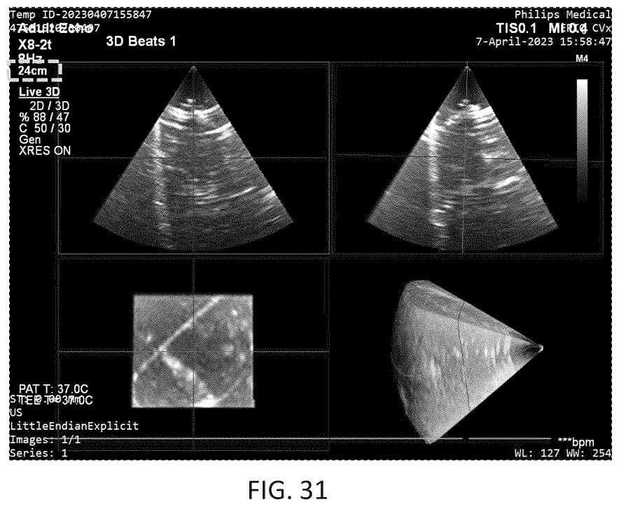

Referring now to FIGS. 30A-30C, from the extracted intersection lines and the Green image plane pose data, the other image planes may be determined. More specifically, the computer vision module can use the lines that represent the planes in each image to construct the transforms between the Red and Green Images, and the Blue and Green Images. This can be done based on the illustrated geometry and an understanding of the various echo cross-sections between 3 planes that intersect at a point. Given the measured angles and positions of the intersection lines in each image (each representing the intersection between two image planes), we can calculate the transforms between the images. Note that the transformations will change over time. Hence, once a “Home” pose has been first identified and the probe location with respect to the planes is initially calculated, if the user updates the location of a plane the module should rerun the calculations for the relationship between the planes based on the new angles/positions of the planes in each image. Similarly, the module may use the initial estimated transform between the Green Plane and the probe to update the Green Plane position with respect to the probe based on the new position of the Green plane, and so on. As can be understood with reference to FIG. 31, the computer vision module may use Machine Learning character recognition to recognize the depth value in the original image, and use that depth value to scale the image data shown in the planar images to the size of the actual structures shown therein.

Referring to FIG. 30 and first reviewing the conventions used for determining the image plane poses:
α_{Xyz}=angle between y and z in the X plane
θ_{Xy}=angle between horizontal (0°) and y in the X plane
\vec{n}_{X}=normal vector to the X plane (e.g. G, R, B) that defines the plane
\vec{n}_{YX}=normal vector that describes the intersection between Y and Z planes
Y_{Xy}=tilt angle about \vec{n}_{XY} that describes the plane Y
For:

$${γ}_{Gb} , {γ}_{Gr}$$

$${\vec{n}}_{G} = [ \begin{matrix}0 \\ 0 \\ 1\end{matrix} ] {\vec{n}}_{GB} = [ \begin{matrix}cos {θ}_{Gb} \\ sin {θ}_{Gb} \\ 0\end{matrix} ] {\vec{n}}_{GR} = [ \begin{matrix}cos {θ}_{Gr} \\ sin {θ}_{Gr} \\ 0\end{matrix} ]$$

Angle between planes at each plane: α_{Gbr}, α_{Rbg}, α_{Bgr}
All planes cross through point (0, 0, 0)
   Now reviewing an approach to these calculations, if we set coordinates based on the green plane, we know:
   The green plane equation is

$$0 x + 0 y + 1 z = 0 , or -> {\vec{n}}_{G} = [ \begin{matrix}0 \\ 0 \\ 1\end{matrix} ]$$

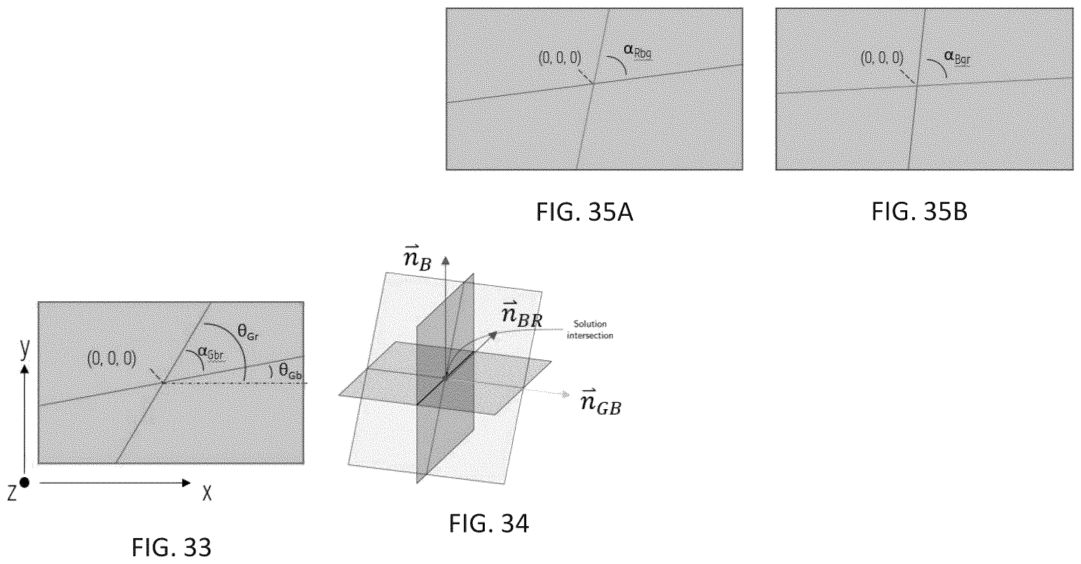

We also know the normal vectors to the lines that intersect the green planes, as shown in FIG. 33, as follows:

$${\vec{n}}_{GB} = [ \begin{matrix}cos {θ}_{Gb} \\ sin {θ}_{Gb} \\ 0\end{matrix} ] {\vec{n}}_{GR} = [ \begin{matrix}cos {θ}_{Gr} \\ sin {θ}_{Gr} \\ 0\end{matrix} ]$$

3 planes that cross at some point, will create 3 lines (the intersection between each pair of planes);
2 of those lines (vectors) will lie on each plane, as shown in FIG. 34
To define the Red and Blue planes in the original green plane coordinate space, we calculate the cross product between the two lines that intersect between that plane and the two others
We already have 1 vector for each plane, n\vec{ }_GB for the blue plane, n\vec{ }_GR for the red plane
The third vector intersection between planes, n [symbol] _BR can be used for both planes. We want to solve for n [symbol] _BR
Referring now to FIGS. 35A and 35B, and knowing:

$$cos θ = \frac{\vec{u} \cdot \vec{v}}{‖ \vec{u} ‖ \cdot ‖ \vec{v} ‖}$$

[symbol] _{BR} is not represented in the green image (i.e. the green coordinate frame)
We can solve for it using angle relationship between vectors, and the two angles
(α_{Rbg} and α_{Rbg}) that [symbol] _{BR} makes with the Green plane. We get two equations:

$$\begin{matrix}{\vec{n}}_{RB} \cdot {\vec{n}}_{RG} = cos ( {α}_{Rbg} ) \vert {\vec{n}}_{RB} \vert \vert {\vec{n}}_{RG} \vert \\ {\vec{n}}_{RB} \cdot {\vec{n}}_{GB} = cos ( {α}_{Bgr} ) \vert {\vec{n}}_{RB} \vert \vert {\vec{n}}_{GB} \vert \\ {\vec{n}}_{RB} \cdot {\vec{n}}_{RG} = cos ( {α}_{Rbg} ) \vert {\vec{n}}_{RB} \vert \vert {\vec{n}}_{RG} \vert \\ {\vec{n}}_{RB} \cdot {\vec{n}}_{GB} = cos ( {α}_{Bgr} ) \vert {\vec{n}}_{RB} \vert \vert {\vec{n}}_{GB} \vert\end{matrix}$$

We know | [symbol] _{XY}|=1, so we can eliminate
We know

$${\vec{n}}_{GB} = [ \begin{matrix}cos {θ}_{Gb} \\ sin {θ}_{Gb} \\ 0\end{matrix} ] and {\vec{n}}_{GR} = [ \begin{matrix}cos {θ}_{Gr} \\ sin {θ}_{Gr} \\ 0\end{matrix} ]$$

So we get the following system of equations:

$$\begin{matrix}X cos {θ}_{Gr} + Y sin {θ}_{Gr} = cos ( {α}_{Rbg} ) \\ X cos {θ}_{Gb} + Y sin {θ}_{Gb} = cos ( {α}_{Bgr} ) \\ Z = \sqrt{1 - {X}^{2} - {Y}^{2}}\end{matrix} OR$$

$$[ \begin{matrix}cos {θ}_{Gr} & sin {θ}_{Gr} \\ cos {θ}_{Gb} & sin {θ}_{Gb}\end{matrix} ] [ \begin{matrix}X \\ Y\end{matrix} ] = [ \begin{matrix}cos ( {α}_{Rbg} ) \\ cos ( {α}_{Bgr} )\end{matrix} ] Z = \sqrt{1 - {X}^{2} - {Y}^{2}}$$

$$Where \vert {n}_{Rb} \vert = [ \begin{matrix}X \\ Y \\ Z\end{matrix} ]$$

Referring again to FIG. 34:
Then to get the equations of the planes w/rt to the Green coordinate frame:

$$\begin{matrix}{\vec{n}}_{R} = \vert {\vec{n}}_{RB} \vert \times \vert {\vec{n}}_{RG} \vert \\ {\vec{n}}_{B} = \vert {\vec{n}}_{RB} \vert \times \vert {\vec{n}}_{BG} \vert\end{matrix}$$

$$Equation of Green Plane \to {\vec{n}}_{G} \cdot [ \begin{matrix}X \\ Y \\ Z\end{matrix} ] = 0 or [ \begin{matrix}0 \\ 0 \\ 1\end{matrix} ] \cdot [ \begin{matrix}X \\ Y \\ Z\end{matrix} ] = 0$$

$$Equation of Red Plane \to {\vec{n}}_{R} \cdot [ \begin{matrix}X \\ Y \\ Z\end{matrix} ] = 0$$

$$Equation of Blue Pane \to {\vec{n}}_{B} \cdot [ \begin{matrix}X \\ Y \\ Z\end{matrix} ] = 0$$

$$where [ \begin{matrix}X \\ Y \\ Z\end{matrix} ] is a point in green coordinate space$$

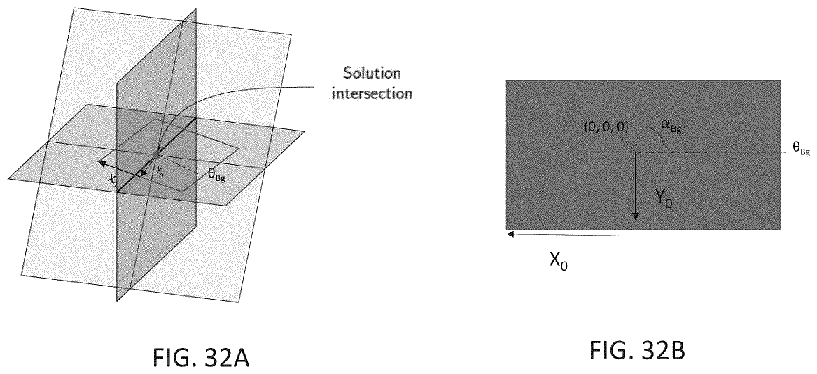

Referring to FIGS. 32A and 32B, with these plane orientations and positions, we can locate the images on those planes.

While the exemplary embodiments have been described in some detail for clarity of understanding and by way of example, a variety of modifications, changes, and adaptations of the structures and methods described herein will be obvious to those of skill in the art. Hence, the scope of the present invention is limited solely by the claims attached hereto.

What is claimed is:

1. A robot system for aligning a diagnostic or therapeutic robot with a target tissue in a three-dimensional (3D) workspace inside a patient body, the system for use with an ultrasound system including a transducer for generating three-dimensional (“3D”) image data of the 3D workspace, a display for showing a plurality of planar images of the 3D workspace, an input, and a multi-plane reconstruction (“MPR”) module coupling the transducer and the input to the display so that the planar images on the display show the 3D data adjacent associated imaging planes, and so as to facilitate manipulation of the imaging planes using the input, wherein an intersection of the imaging planes defines an image-based position and orientation within the 3D workspace, the robot system comprising:
an articulating arm and a proximal driver;
the articulating arm having a proximal end and a distal end with an axis therebetween, the proximal end couplable with the driver, the distal end configured for insertion into the 3D workspace; and
the driver couplable with the processor of the ultrasound system so as to induce driving of the distal end of the articulating arm toward the image-based position and orientation in response to the user manipulating the input of the ultrasound system.

2. The robot system of claim 1, further comprising a therapeutic or diagnostic tool supported by the distal end, and an augmented reality (AR) module coupling the processor of the imaging system with the display, the AR module configured to superimpose an image of a virtual therapeutic or diagnostic tool on the display at the image-based position and orientation, wherein the processor induces driving of the tool, as shown in the display, into alignment with the virtual tool when the robotic arm is advanced axially into the patient.

3. The robot system of claim 1, wherein the robotic arm comprises a robotically articulated catheter, and wherein the transducer comprises a transesophageal echocardiography (“TEE”) or intracardiac echocardiography (“ICE”) transducer.

4. The robot system of claim 3, wherein the robotic articulated catheter is configured for use within the cardiovascular system and supports, near the distal end, a tool configured to performing a transcatheter interventional therapy.

5. The robot system of claim 4, wherein the tool comprises a transcatheter edge-to-edge repair (TEER) clip.

6. The robot system of claim 1, the ultrasound system generating an image data stream during use, the robot system further comprising:
a module coupled with the driver and configured to determine a pose of the transducer in the 3D workspace;
an image processing module coupling the processor of the ultrasound system with the driver and configured to identify poses of the image planes relative to the transducer in response to the image data stream, the driver inducing movement of the distal end in response to the pose of the transducer and the poses of the image planes during use.

7. The robot system of claim 4, further comprising:
a catheter trajectory planning module configured to display an AR catheter body extending from a guide sheath to the tool,
a trajectory modification input for altering an AR axis of the AR catheter body, and
an insertion control module for varying a shape of the catheter body axis so as to induce following of the AR axis by the distal end during axial advancement of the catheter toward the target tissue.

8. The robot system of claim 2, wherein the robotic articulated catheter supports the ICE transducer.

9. A robot system for commanding movement of a robot system in a three-dimensional (3D) workspace shown in a display, the robot system comprising:
an elongate body having a proximal end and a distal end with an axis therebetween; a driver couplable with the proximal end of the elongate body;
a processor couplable to the driver, the processor configured for receiving, relative to a first image plane, a first command to move a second image plane shown in the display, the first image plane and second image planes being disposed in the 3D workspace and shown in a display with the first image plane extending along an associated first window of the display and the second image plane extending along a second window of the display;
the processor also configured for receiving, relative to the second image plane, a second command to move the first image plane; and
the processor configured for determining a line of intersection between the first image plane and the second image plane in the 3D workspace, and for transmitting a robot movement command to the driver so that the axis of the body moves toward alignment with the line of intersection in the 3D workspace.

10. A method for aligning a diagnostic or therapeutic robot with a target tissue in a three-dimensional (3D) workspace inside a patient body, the method comprising:
generating three-dimensional (“3D”) image data of the 3D workspace;
showing a plurality of planar images of the 3D workspace on a display;
coupling the transducer to the display so that the planar images on the display show the 3D data adjacent associated imaging planes;
manipulating the imaging planes using the input, wherein an intersection of the imaging planes defines an image-based position and orientation within the 3D workspace; and
inducing driving of a distal end of an articulating arm in the 3D workspace toward the image-based position and orientation in response to the user manipulating the input of the imaging system.

11. The method of claim 10, further comprising superimposing an Augmented Reality (“AR”) image of a virtual therapeutic or diagnostic tool on the display at the image-based position and orientation, wherein the driving of the distal end comprises driving of a therapeutic or diagnostic tool supported by the distal end, as shown in the display, into alignment with the virtual tool, as shown in the display, when the robotic arm is advanced axially into the patient.

12. A method for commanding movement of a robot system in a three-dimensional (3D) workspace shown in a display, the robot system having a body with an axis, the method comprising:
receiving, with a processor and relative to a first image plane, a first command to move a second image plane shown in the display, the first image plane and second image planes being disposed in the 3D workspace and shown in the display with the first image plane extending along an associated first window of the display and the second image plane extending along a second window of the display;
receiving, with the processor and relative to the second image plane, a second command to move the first image plane;
determining, with the processor, a line of intersection between the first image plane and the second image plane in the 3D workspace; and
transmitting, from the processor, a robot movement command to the robot so that the axis of the body moves toward alignment with the line of intersection in the 3D workspace.

13. The method of claim 12, wherein the robot system includes a therapeutic or diagnostic tool supported by a distal end of the body, and further comprising superimposing an image of a virtual therapeutic or diagnostic tool on the display with the axis aligned at the line of intersection, wherein the robot movement command induces driving of the tool, as shown in the display, into alignment with the virtual tool, as shown in the display, when the robotic arm is advanced axially into the patient.

14. The method of claim 12, the robot system including 3D image data from the 3D workspace, the second command to move the first image plane inducing reconstruction of a first 2D image along the first image plane from the 3D image data, the first 2D image being shown in the first window of the display, the first command to move the second image plane inducing reconstruction of a second 2D image along the second image plane from the 3D image data, the second 2D image being shown in the second window of the display, the method further comprising:
sensing, with an input device, the first command as entered by a hand of a user, the first command comprising a change, as shown in the second window, to a position of the line of intersection or a change to an orientation of the line of intersection; and
sensing, with the input device, the second command as entered by a hand of a user, the second command comprising a change, as shown in the first window, to a position of the line of intersection or a change to an orientation of the line of intersection.

15. The method of claim 12, further comprising:
receiving, with the processor, a third command to move the first image plane or the second image plane;
wherein the third command is sensed by the input device, the third command comprising a change to a position of the line of intersection or a change to an orientation of the line of intersection, as shown in a third window; and
receiving, with the processor, additional command to move a third image plane relative to the first image plane and the second image plane relative to a third image plane, the third image plane being transverse to the first image plane and the second image plane so as to define an intersection point along the intersection line, the third window comprising a reconstructed image of the 3D image data along the third image plane;
wherein the additional commands are sensed by the input device, the third command comprising a change to a position of the line of intersection or a change to an orientation of the line of intersection, as shown in a third window; and
wherein the robot movement command transmitted by the process is configured to induce movement of an end portion of the body into axial alignment of the end portion with the intersection point.

16. The method of claim 12, wherein the 3D image data comprises 3D ultrasound data generated by an ultrasound machine, and wherein the input device comprises a trackball of the ultrasound machine, the image planes comprising multi-plane reconstruction (MPR) planes.

17. The method of claim 12, wherein the robot system comprises a transcatheter robot system and the body of the robot system comprises a therapeutic or diagnostic tool suitable for use inside a chamber of a heart of a patient.
